(19) United States
(12) Patent Application Publication
(10) Pub. No.: US 2026/0207483 A1
(43) Pub. Date: Jul. 23, 2026

(54) PHARMACEUTICAL COMPOSITIONS COMPRISING NABILONE FOR THE TREATMENT OF OPHTHALMIC CONDITIONS

(71) Applicant: Adtech Pharma, Inc., Warwick, RI (US)

(72) Inventors: Robert J. KUPPER, West Warwick, RI (US); Ping W. CHANG, Waterford, CT (US); Akwete Lex ADJEI, Marco Island, FL (US); Iok-hou PANG, Grand Prairie, TX (US)

(73) Assignee: Adtech Pharma, Inc., Warwick, RI (US)

(21) Appl. No.: 19/142,899

(22) PCT Filed: Dec. 28, 2023
(86) PCT No.: PCT/US2023/086258

§ 371 Date: Jun. 24, 2025

Related U.S. Application Data
(60) Provisional application No. 63/477,698, filed on Dec. 29, 2022.

(51) Int. Cl. A61K 9/00; A61K 9/107; A61K 31/352; A61K 47/02; A61K 47/14; A61K 47/22; A61K 47/44; A61P 27/06

(52) U.S. Cl. CPC A61K 9/0048; A61K 9/107; A61K 31/352; A61K 47/02; A61K 47/14; A61K 47/22; A61K 47/44; A61P 27/06

(57) ABSTRACT

The present disclosure provides compositions comprising at least one compound of Formula (I) that are suitable for application to an ocular surface. The present disclosure also provides methods of making the compositions comprising at least one compound of Formula (I). The present disclosure further provides methods of using the compositions comprising at least one compound of Formula (I) to provide intraocular pressure reduction, neuroprotection, and/or to treat ophthalmic conditions such as glaucoma.

### FIELD OF THE DISCLOSURE

The present disclosure provides compositions comprising at least one compound of Formula (I) that are suitable for application to an ocular surface. The present disclosure also provides methods of making the compositions comprising at least one compound of Formula (I). The present disclosure further provides methods of using the compositions comprising at least one compound of Formula (I) to provide intraocular pressure reduction, neuroprotection, and/or to treat ophthalmic conditions such as glaucoma.

### BACKGROUND

The prevalence of neuropathological ophthalmic conditions is an important public health issue. For example, glaucoma is one of the leading causes of blindness worldwide. In the United States alone, it is estimated that more than 3 million individuals are living with the disease. Glaucoma refers to a group of eye conditions that cause damage to the retina and the optic nerve of the eye. Abnormally high intraocular pressure (IOP) causes damage to the optic nerve which eventually leads to optic nerve degeneration and retinopathy, resulting in vision loss and blindness.

Several lipophilic (and poorly water soluble) drugs have become available in recent years to treat glaucoma and other ophthalmic conditions. For example, isolated compounds from the cannabis plant, such as tetrahydrocannabinol (THC), and other modulators of the cannabinoid receptors, CB1 and CB2, have been shown to reduce IOP and to have neuroprotective and anti-inflammatory properties in the eye, and are therefore useful for the treatment of a variety of ophthalmic diseases (J. Pharm. Sci., 2012, 101(2):616-626; Ophthalmic. Res., 1992, 24:142-149; International J. Pharm., 2010, 393:238-243; United States Patent Publication No. 2016/0184259; U.S. Pat. Nos. 9,265,724; 11,786,463; and Br. J. Ophthalmol., 2004, 88:708-713). However, these, and other, lipophilic drugs present a formulation challenge for scientists because the low aqueous solubility of the drug prohibits preparing eye drop solutions having sufficient drug concentrations delivered for clinical efficacy and safety. Most traditional lipophilic dosage forms for ocular application (e.g., oil solutions, lotions, and gels) are uncomfortable for the patient and do not provide adequate local drug concentrations to the eye. Therefore, low viscosity polar and semipolar formulations presented as eye drops are generally preferred.

For some lipophilic drugs, emulsions can offer significant advantages, including increased solubility of the active ingredient, prolonged local activity, and improved ocular bioavailability. However, the design of emulsion formulations that are biologically compatible, stable, and sterilizable remains a challenge.

Thus, new or improved ophthalmic drug delivery systems are continually needed that are stable, well-tolerated, have enhanced activity, and possess other advantageous features.

### BRIEF SUMMARY

The present invention is directed to a composition suitable for application to an ocular surface, comprising:
at least one compound of Formula (I)

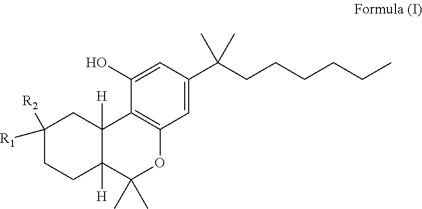

where R₁ and R₂ together form ═O or R₁ is —OH and R₂ is —H;
an oil;
a surfactant; and
water,
   wherein the composition comprises a continuous phase comprising water; and a dispersed phase comprising a plurality of nanosized particles of at least one compound of Formula (I), droplets of the oil, or a combination thereof, and wherein at least about 60% of the at least one compound of Formula (I) is in the dispersed phase.

In some embodiments, the composition is a dispersion; a suspension; an emulsion; or any combination thereof.

In some embodiments, the compound of Formula (I) is selected from the group consisting of:

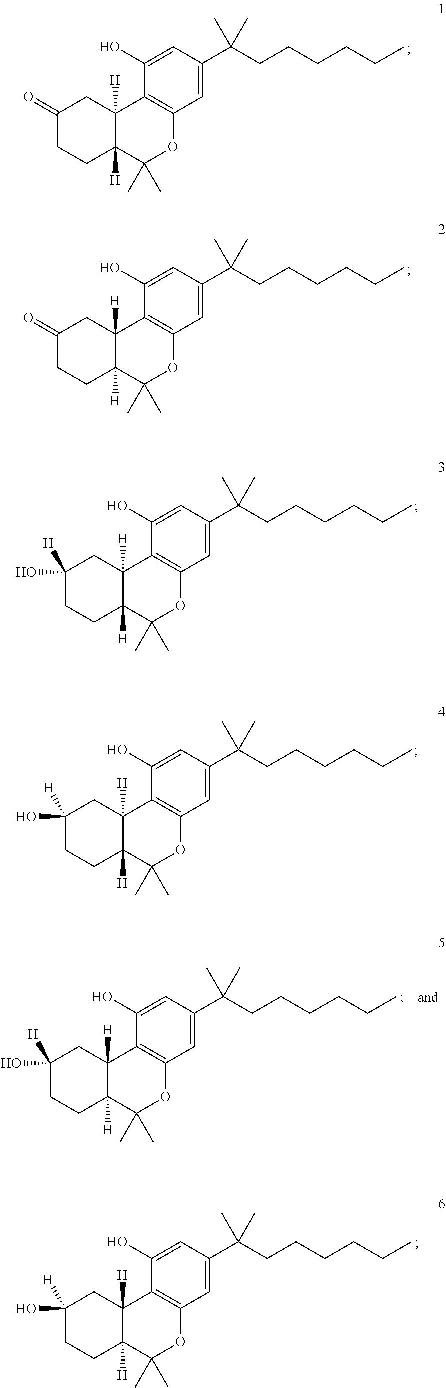 [SMILES: CCCCCCC(C)(C)c1cc(O)c2c(c1)OC(C)(C)[C@@H]1CCC(=O)C[C@@H]21 ; CCCCCCC(C)(C)c1cc(O)c2c(c1)OC(C)(C)[C@H]1CCC(=O)C[C@H]21 ; CCCCCCC(C)(C)c1cc(O)c2c(c1)OC(C)(C)[C@@H]1CC[C@H](O)C[C@@H]21 ; CCCCCCC(C)(C)c1cc(O)c2c(c1)OC(C)(C)[C@@H]1CC[C@@H](O)C[C@@H]21 ; CCCCCCC(C)(C)c1cc(O)c2c(c1)OC(C)(C)[C@H]1CC[C@H](O)C[C@H]21 ; CCCCCCC(C)(C)c1cc(O)c2c(c1)OC(C)(C)[C@H]1CC[C@@H](O)C[C@H]21]

In some embodiments, the compound of Formula (I) is

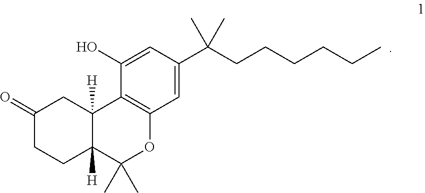 [SMILES: CCCCCCC(C)(C)c1cc(O)c2c(c1)OC(C)(C)[C@@H]1CCC(=O)C[C@@H]21]

In some embodiments, the compound of Formula (I) is

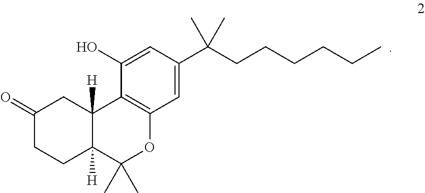 [SMILES: CCCCCCC(C)(C)c1cc(O)c2c(c1)OC(C)(C)[C@H]1CCC(=O)C[C@H]21]

In some embodiments, the compound of Formula (I) is a racemic mixture of

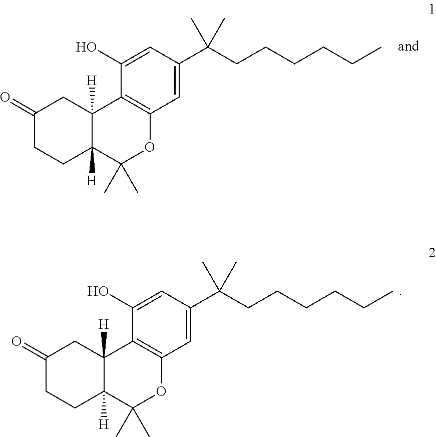 [SMILES: CCCCCCC(C)(C)c1cc(O)c2c(c1)OC(C)(C)[C@@H]1CCC(=O)C[C@@H]21 ; CCCCCCC(C)(C)c1cc(O)c2c(c1)OC(C)(C)[C@H]1CCC(=O)C[C@H]21]

In some embodiments, the compound of Formula (I) is

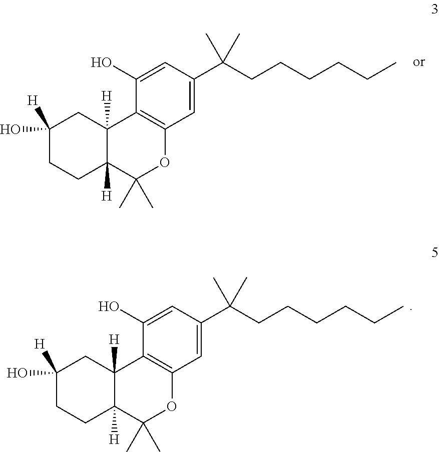 [SMILES: CCCCCCC(C)(C)c1cc(O)c2c(c1)OC(C)(C)[C@@H]1CC[C@H](O)C[C@@H]21 ; CCCCCCC(C)(C)c1cc(O)c2c(c1)OC(C)(C)[C@H]1CC[C@H](O)C[C@H]21]

In some embodiments, at least about 90 weight % of the compound in the composition is present after the composition is stored at a condition selected from the group consisting of at least two years at about 5° C.; and at least one month at about 25° C.

In some embodiments, the oil and water are present in the composition in a ratio (w/w) of about 1:1 to about 1:1000.

In some embodiments, the composition is a suspension or an emulsion.

In some embodiments, the composition is a microemulsion or a nanodispersion.

In some embodiments, at least about 90% of the droplets of the oil in the composition have a mass median diameter is less than about 500 nm, for example less than 500 nm. In some embodiments, at least about 90% of the droplets of the oil in the composition have a mass median diameter of less than about 350 nm.

In some embodiments, at least about 90% of the droplets of the oil in the composition have a mass median diameter of between about 100 nm to about 200 nm.

In some embodiments, at least about 90% of the at least one compound of Formula (I) in the composition has a mass median diameter of less than about 500 nm.

In some embodiments, at least about 90% of the at least one compound of Formula (I) in the composition has a mass median diameter of less than about 350 nm.

In some embodiments, at least about 90% of the at least one compound of Formula (I) in the composition has a mass median diameter of between about 100 nm to about 200 nm.

In some embodiments, the composition comprises about 0.005 weight % to about 0.5 weight % of the compound of Formula (I).

In some embodiments, the oil is selected from the group consisting of sesame oil, safflower oil, linseed oil, castor oil, a medium chain diglyceride ester oil, a medium chain triglyceride ester oil, soybean oil, olive oil, cotton seed oil, peanut oil, mineral oil, and combinations thereof.

In some embodiments, the oil is sesame oil, safflower oil, castor oil, or linseed oil. In some embodiments, the oil is safflower oil.

In some embodiments, the oil is mineral oil.

In some embodiments, the oil is castor oil.

In some embodiments, the composition comprises about 1.5 weight % to about 25.0 weight % oil.

In some embodiments, the surfactant is selected from the group consisting of polyoxyethylene (20) sorbitan monooleate (TWEEN® 80); polyoxyethylene (20) sorbitan monolaurate (TWEEN® 20); 4-(1,1,3,3-tetramethylbutyl) phenol polymer with formaldehyde and oxirane (Tyloxapol); sorbitan monooleate (Span 80); polyoxyethylated 12-hydroxystearic acid (KOLLIPHER® HS 15); polyoxyl 35 castor oil; polyoxyl 40 hydrogenated castor oil; polyoxyl 40 stearate; polysorbate 80; and combinations thereof.

In some embodiments, the surfactant is polyoxyethylene (20) sorbitan monooleate (TWEEN® 80).

In some embodiments, the composition comprises about 0.5 weight % to about 5 weight % surfactant.

In some embodiments, the composition further comprises a co-solvent.

In some embodiments, the composition further comprises a co-solvent, wherein the co-solvent is glycerin.

In some embodiments, the composition comprises about 1 weight % to about 10 weight % co-solvent.

In some embodiments, the composition further comprises at least one pH adjusting agent.

In some embodiments, the composition further comprises at least one pH adjusting agent, wherein the at least one pH adjusting agent is sodium hydroxide or hydrochloric acid.

In some embodiments, the composition further comprises at least one pH adjusting agent, wherein the at least one pH adjusting agent is sodium hydroxide and hydrochloric acid.

In some embodiments, the composition has a pH of about 6.5 to about 7.5.

In some embodiments, the composition further comprises a stabilizing agent.

In some embodiments, the composition further comprises a stabilizing agent, wherein the stabilizing agent is an antioxidant.

In some embodiments, the composition further comprises a stabilizing agent, wherein the stabilizing agent is an antioxidant selected from the group consisting of α-tocopherol acetate, ascorbic acid, butylated hydroxytoluene (BHT), butylated hydroxyanisole (BHA), monothioglycerol, bisulfites, and combinations thereof.

In some embodiments, the composition further comprises a stabilizing agent, wherein the stabilizing agent is an antioxidant, and wherein the antioxidant is selected from the group consisting of BHT, BHA, and combinations thereof.

In some embodiments, the composition has an osmolarity of about 250 mOsm/L to about 330 mOsm/L.

In some embodiments, the composition is adapted to be permeable through a cornea of a patient to provide a therapeutically effective concentration of the at least one compound of Formula (I).

In some embodiments, the composition comprises
about 0.005 weight % to about 0.5 weight % of the compound of Formula (I);
about 5 weight % to about 25 weight % of an oil selected from the group consisting of sesame oil, castor oil, mineral, and combinations thereof;
about 1.5 weight % to about 2.5 weight % polyoxyethylene (20) sorbitan monooleate (TWEEN® 80);
about 0.01 weight % to about 1.25 weight % of BHT;
about 0.01 weight % to about 1.25 weight % of BHA;
about 2.5 weight % glycerin; and
water.

The present invention also discloses a method of treating or preventing an ophthalmic condition in a subject identified in need of such treatment, the method comprising administering to at least one eye of the subject in need thereof, a therapeutically effective amount of the composition disclosed herein.

In some embodiments, the composition is administered once daily.

In some embodiments, the composition is administered twice daily.

In some embodiments, the composition is administered topically.

In some embodiments, the composition is administered topically as liquid drops, a liquid wash, a gel, an ointment, a spray, or a combination thereof.

In some embodiments, the composition is applied as one or more drops to an ocular surface.

In some embodiments, the ophthalmic condition is selected from the group consisting of glaucoma, age-related macular degeneration (AMD), ophthalmitis, and conjunctivitis.

In some embodiments, the ophthalmic condition is glaucoma.

In some embodiments, the method decreases intraocular pressure (IOP) for a period of at least about 1 hour after administering the composition to the eye.

In some embodiments, the method decreases intraocular pressure (IOP) for a period of at least about 4 hours after administering the composition to the eye.

The present invention also discloses a method of preparing the composition disclosed herein, comprising:
combining the compound and the oil to form an oil phase;
combining the surfactant and water to form an aqueous phase;
combining the oil phase and the aqueous phase to form a premix;
homogenizing the premix to form a homogenized premix; and
microfluidizing the homogenized premix to provide the composition.

In some embodiments, the oil phase is prepared at a temperature from about 50° C. to about 100° C.

In some embodiments, the aqueous phase is prepared at a temperature from about 50° C. to about 100° C.

In some embodiments, the premix is homogenized at a speed of about 5000 rpm to about 15000 rpm.

In some embodiments, the premix is homogenized for about 2 minutes to about 20 minutes.

In some embodiments, the homogenized premix is microfluidized at a temperature from about 15° C. to about 50° C.; followed by chilling at a temperature from about 15° C. to about 30° C. using chilled water circulating in the jacket of a mixing vessel.

In some embodiments, the premix is microfluidized at a pressure from about 10,000 PSI to about 20,000 PSI.

In some embodiments, the method of preparing further comprises adjusting the pH of the homogenized premix to about 6.5 to about 7.5.

In some embodiments, the droplets of the oil comprise at least one compound of Formula (I) dissolved in the oil.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

The present invention will now be described with reference to the accompanying drawings. In the drawings, like reference numbers indicate identical or functionally similar elements.

FIG. 1 is a graph comparing nabilone solubilities in oils (“supernatant”) applicable to topical ophthalmic formulations. “Bulk Form I” is commercially available nabilone (Purysis LLC, Athens, GA). “NF Crystalline” is “Bulk Form I” that has been nanosized to a less than 500 median mass diameter (MMD) particle size.

FIG. 2 is a graph showing the average droplet size (Z Avg) and polydispersity index (PDI) of samples described in Example 1 of a nabilone nanoemulsion (0.01 weight %) collected in 12 minute increments (Cycles 1-5) during high shear homogenization using an appropriate homogenizer such as a MICROFLUIDIZER® (Microfluidics, Westwood, MA).

FIG. 3 is a graph showing the size distribution by intensity of a sample described in Example 1 of a nabilone nanoemulsion (0.01 weight %) collected at a particular time period (Cycle 3) during microfluidization.

FIG. 4 is a graph showing the size distribution by intensity of a sample described in Example 1 of a nabilone nanoemulsion (0.01 weight %) collected at a particular time period (Cycle 5) during microfluidization.

FIG. 5 is a graph showing the size distribution by time period of samples described in Example 1 of a nabilone nanoemulsion (0.01 weight %) collected at various time periods (Cycles 1-5) during microfluidization. A D (10) value means that 10% of the particles in the composition are smaller than the indicated size. A D (50) value means that 50% of the particles in the composition are smaller than the indicated size. A D (90) value means that 90% of the particles in the composition are smaller than the indicated size. And, a D (99) value means that 99% of the particles in the composition are smaller than the indicated size.

FIG. 6 is a graph showing the average droplet size (Z Avg) and polydispersity index (PDI) of samples described in Example 3 of a nabilone nanoemulsion (0.05 weight %) collected in about 12 minute increments (Cycles 1-5) during microfluidization.

FIG. 7 is a graph showing the size distribution by intensity of samples described in Example 3 of a nabilone nanoemulsion (0.05 weight %) collected at a particular time period (Cycle 3) during microfluidization.

FIG. 8 is a graph showing the size distribution by intensity of samples described in Example 3 of a nabilone nanoemulsion (0.05 weight %) collected at a particular time period (Cycle 5) during microfluidization.

FIG. 9 is a graph showing the size distribution by time period of samples described in Example 3 of a nabilone nanoemulsion (0.05 weight %) collected at various time periods (Cycles 1-5) during microfluidization.

FIG. 10 is a graph comparing the intraocular pressure (IOP) measured in untreated control mice eyes and in mice eyes treated with the nabilone composition (0.01 weight %) described in Example 1 for 48 hours after dosing.

FIG. 11 is a graph comparing the intraocular pressure (IOP) measured in untreated control mice eyes and in mice eyes treated with the nabilone composition (0.05 weight %) described in Example 3 for 48 hours after dosing.

FIG. 12 is a graph comparing the intraocular pressure (IOP) measured in untreated control mice eyes and in mice eyes treated with a timolol composition (0.5 weight %) for 48 hours after dosing.

FIG. 13 is a graph comparing the intraocular pressure (IOP) measured in untreated control mice eyes and in mice eyes treated with the control composition described in Example 4 (vehicle for nabilone nanoemulsion (0.05 weight %)) for 48 hours after dosing.

FIG. 14 is a graph comparing the intraocular pressure (IOP) measured in untreated mice eyes and in mice eyes treated with the nabilone nanoemulsion (0.025 weight %) for 48 hours after dosing.

FIG. 15 is a graph comparing the intraocular pressure (IOP) in untreated mice eyes and in mice eyes treated with the nabilone nanoemulsion (0.075 weight %) for 48 hours after dosing.

FIG. 16 is a composite graph showing the intraocular pressure (IOP) in untreated mice eyes (“Untreated Naive for Nabilone” 0.01%, 0.025%, 0.05%, and 0.075% (by weight)) and in mice eyes treated with the nabilone nanoemulsions (0.01%, 0.025%, 0.05%, and 0.075% (by weight)), with the corresponding placebo (“Untreated Naive Timolol” 0.5 weight %), and with timolol (0.5 weight %).

FIG. 17 is a graph showing the dose response of nabilone nanoemulsions (“Nab” 0.01%, 0.025%, 0.05%, and 0.075% (by weight)) as a percent of intraocular pressure (IOP) reduction 6 hours after dosing compared to a placebo vehicle (“Vehicle”) and timolol (0.5 weight %).

FIG. 18 is a graph showing the dose response of nabilone nanoemulsions (“Nab” 0.01%, 0.025%, 0.05%, and 0.075% (by weight)) as a percent of intraocular pressure (IOP) reduction 24 hours after dosing compared to a placebo vehicle (“Vehicle”) and timolol (0.5 weight %).

### DETAILED DESCRIPTION

As used above, and throughout the description, the following terms, unless otherwise indicated, shall be understood to have the following meanings.

Unless stated otherwise, the terms “a” and “an” and “the” and similar references used in the context of describing a particular aspect of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

It is understood that wherever aspects are described herein with the language “comprising,” otherwise analogous aspects described in terms of “consisting of” and/or “consisting essentially of” are also provided.

The term “about” encompasses the range of experimental error that occurs in any measurement. The term “about” as used in connection with a numerical value throughout the specification and the claims denotes an interval of accuracy, familiar and acceptable to a person skilled in the art. In general, such interval of accuracy is +10%. Thus, “about ten” means 9 to 11. All numbers in this description indicating amounts, ratios of materials, physical properties of materials, and/or use are to be understood as modified by the word “about,” except as otherwise explicitly indicated.

The “weight percentage” or “wt %” or “weight %” as used herein refers to the weight percentage of an ingredient as compared to the total weight of the composition. For example, the weight percentage of nabilone refers to the weight percentage of the active nabilone in the composition.

The phrase “mass median diameter” as used herein refers to a particle diameter at which 50% of the particle mass (e.g., droplets of an oil, solid particles) is contained in smaller particles and 50% is contained in larger particles. The phrase mass median diameter can be abbreviated as MMD.

Nabilone, sold as a polymorphic crystalline powder in capsules for oral administration under the brand name of CESAMET®, is a synthetic cannabinoid. Nabilone is a 1:1 racemic mixture of 6aR, 10aR ((6aR,10aR)-1-hydroxy-6,6-dimethyl-3-(2-methyl-2-octanyl)-6,6a,7,8,10,10a-hexahydro-9H-benzo[c]chromen-9-one) and 6aS, 10aS ((6aS,10aS)-1-hydroxy-6,6-dimethyl-3-(2-methyl-2-octanyl)-6,6a,7,8,10,10a-hexahydro-9H-benzo[c]chromen-9-one) optical isomers.

In the Summary of Basis of Approval for NDA 18-677 approved on Dec. 26, 1985, an oral dose of CESAMET® was studied for the treatment of glaucoma. The study contained 17 patients. During the study, 5% of the 17 patients reported experiencing 78.9% of the adverse events with five serious adverse events being reported. All but one of the 5% of patients experienced the adverse events after a single dose. A notable significant adverse event was the significant blood pressure drop (hypotension) experienced by patients with one patient becoming nauseous and fainting after a single dose. Due to the prevalence of adverse events, the study was discontinued.

Nabilone is difficult to solubilize in most solvents such that preparing a liquid composition comprising nabilone is very challenging. TABLE 1 includes solvents in which nabilone is freely soluble. These solvents are not suitable for use in human pharmaceutical formulations. Nabilone is insoluble in water at any pH based on USP classification of solubility: freely soluble is >100 mg/mL; soluble is 33-100 mg/mL; sparingly soluble is 10-33 mg/mL; slightly soluble is 1-10 mg/mL; very slightly soluble is 0.1-1 mg/mL; and practically insoluble is <0.1 mg/mL.

TABLE 1
Solubility of Nabilone in Common Solvents

| Solvent | USP Classification |
|---|---|
| Dimethylformamide | Freely Soluble |
| Tetrahydrofuran | |
| Dimethyl sulfoxide | |
| 2-Methyltetrahydrofuran | |
| Dichloromethane | |
| Dimethoxyethane | |
| 1,4-Dioxane | |
| Methyl ethyl ketone | |
| p-Xylene | |
| Acetone | |

It is also challenging to solubilize nabilone in pharmaceutically acceptable oils, even at elevated temperatures. As shown in TABLE 2, only a very small amount of nabilone dissolves in refined sesame oil, even after heating to 90° C., with the majority of the nabilone precipitating at the bottom.

TABLE 2
Solubility of Nabilone in Sesame Oil

| | Obser- | Observation | | Nabilone |
|---|---|---|---|---|
| | vation | After | Nabilone | recovered |
| | Before | Centrifuge | added | from oil phase |
| Sample | Centri- | (14000 rpm, | (w/w in | (w/w in |
| Conditions | fuge | 10 minutes) | mg/g) | mg/mL) |
| 5% w/w of | Turbid | Clear solution | 250 mg/g | 6.3 |
| nabilone in | viscous | with sample | | |
| sesame oil; | liquid | precipitate at | | |
| heated to 90° C. | | bottom | | |
| for 3 hours | | | | |
| 5% w/w of | Turbid | Clear solution | 250 mg/g | 3.9 |
| nabilone in | viscous | with sample | | |
| sesame oil | liquid | precipitate at | | |
| | | bottom | | |

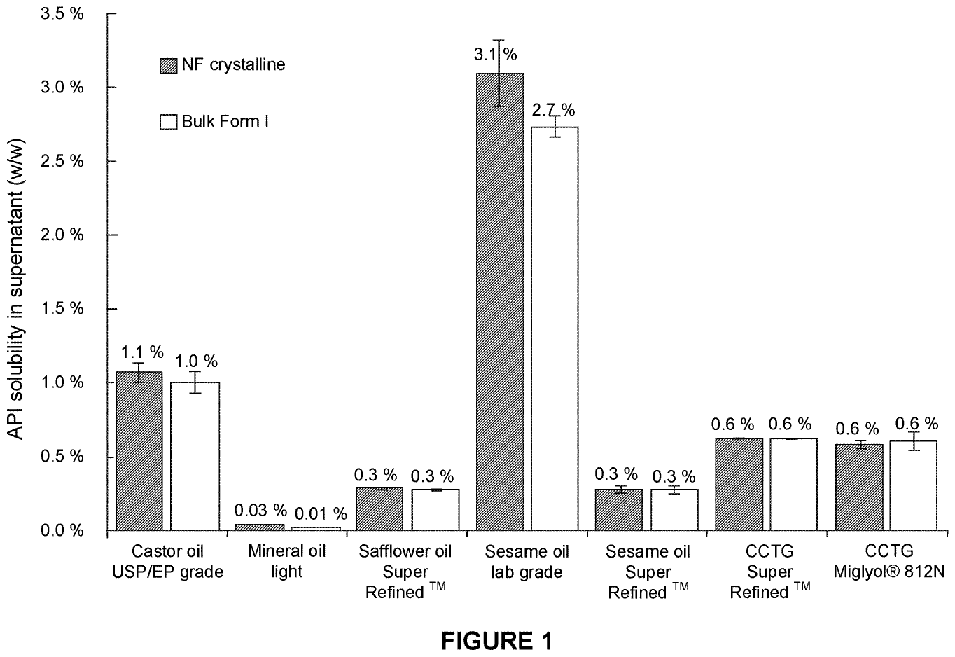

Nabilone solubility in other oils applicable to topical ophthalmic formulations are summarized in FIG. 1, wherein “Bulk Form I” is a typical lot of nabilone (Purisys LLC, Athens, GA) and “NF crystalline” is nanosized from Bulk Form I.

As described herein, the disclosed compositions are suitable for topical administration of at least one compound of Formula (I) to an ocular surface. The compositions are stable, well-tolerated, and capable of delivering therapeutically effective amounts of at least one compound of Formula (I) to target sites, including sites on the surface of and/or within the eye. Surprisingly, the compositions are physically, chemically, and/or microbiologically stable and exhibit efficacious and prolonged intraocular pressure (IOP) lowering effects.

The present invention provides a composition suitable for application to an ocular surface, comprising:
at least one compound of Formula (I)

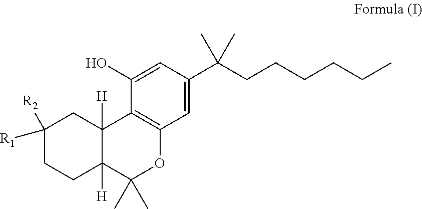

where R₁ and R₂ together form ═O or R₁ is —OH and R₂ is —H;
an oil;
a surfactant; and
water,
   wherein the composition comprises a continuous phase comprising water; and a dispersed phase comprising a plurality of nanosized particles of at least one compound of Formula (I), droplets of the oil, or a combination thereof, and wherein at least about 60% of the at least one compound of Formula (I) is in the dispersed phase.

The present invention also provides a composition suitable for application to an ocular surface, comprising:
at least one compound of Formula (I)

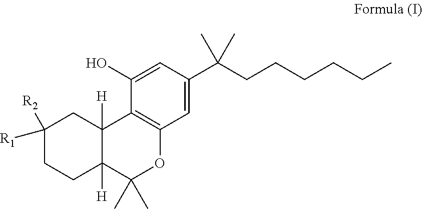

where R₁ and R₂ together form ═O or R₁ is —OH and R₂ is —H;
an oil;
a surfactant;
optionally a co-solvent;
optionally a pH adjusting agent;
optionally a buffering agent;
optionally a stabilizing agent; and
water,
   wherein the composition comprises a continuous phase comprising water; and a dispersed phase comprising a plurality of nanosized particles of at least one compound of Formula (I), droplets of the oil, or a combination thereof, and wherein at least about 60% of the at least one compound of Formula (I) is in the dispersed phase.

### Compound of Formula (I)

In some embodiments, in the at least one compound of Formula (I), R₁ and R₂ together form ═O.

In some embodiments, in the at least one compound of Formula (I), R₁ is —OH and R₂ is —H.

In some embodiments, the at least one compound of Formula (I) is selected from the group consisting of:

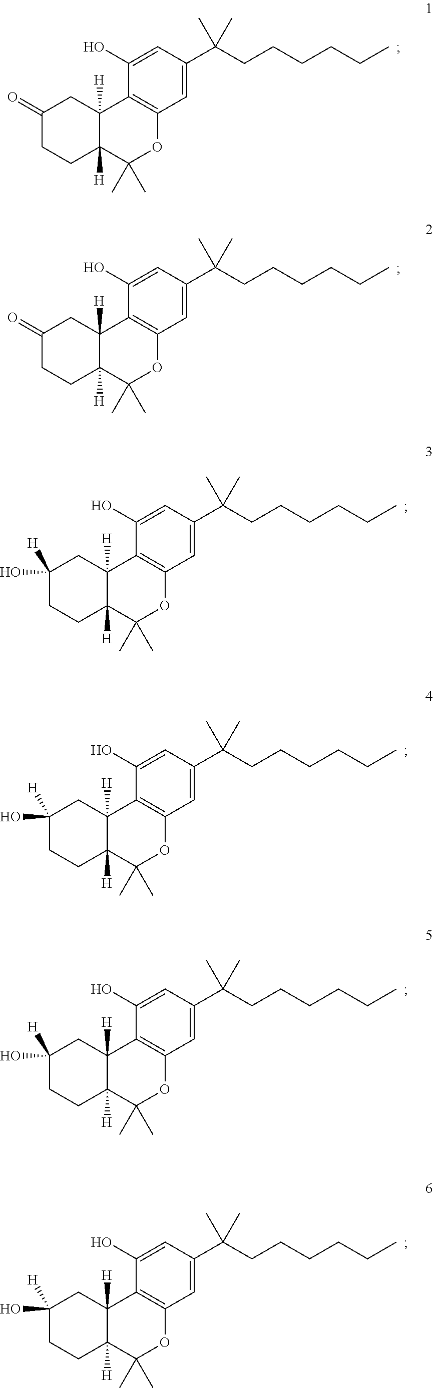 [SMILES: CCCCCCC(C)(C)c1cc(O)c2c(c1)OC(C)(C)[C@@H]1CCC(=O)C[C@@H]21 ; CCCCCCC(C)(C)c1cc(O)c2c(c1)OC(C)(C)[C@H]1CCC(=O)C[C@H]21 ; CCCCCCC(C)(C)c1cc(O)c2c(c1)OC(C)(C)[C@@H]1CC[C@H](O)C[C@@H]21 ; CCCCCCC(C)(C)c1cc(O)c2c(c1)OC(C)(C)[C@@H]1CC[C@@H](O)C[C@@H]21 ; CCCCCCC(C)(C)c1cc(O)c2c(c1)OC(C)(C)[C@H]1CC[C@H](O)C[C@H]21 ; CCCCCCC(C)(C)c1cc(O)c2c(c1)OC(C)(C)[C@H]1CC[C@@H](O)C[C@H]21]

and combinations thereof.

In some embodiments, the at least one compound of Formula (I) is:

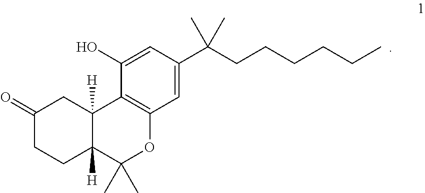 [SMILES: CCCCCCC(C)(C)c1cc(O)c2c(c1)OC(C)(C)[C@@H]1CCC(=O)C[C@@H]21]

In some embodiments, the at least one compound of Formula (I) is:

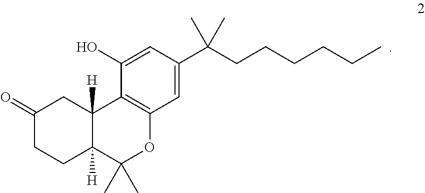 [SMILES: CCCCCCC(C)(C)c1cc(O)c2c(c1)OC(C)(C)[C@H]1CCC(=O)C[C@H]21]

In some embodiments, the at least one compound of Formula (I) is nabilone.

In some embodiments, the compound of Formula (I) is a racemic mixture of:

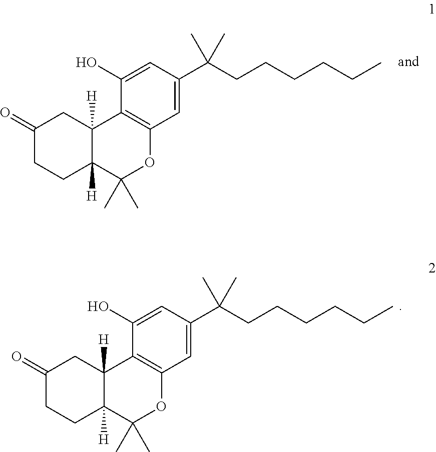 [SMILES: CCCCCCC(C)(C)c1cc(O)c2c(c1)OC(C)(C)[C@@H]1CCC(=O)C[C@@H]21 ; CCCCCCC(C)(C)c1cc(O)c2c(c1)OC(C)(C)[C@H]1CCC(=O)C[C@H]21]

The racemic mixture of Compound 1 and Compound 2 is commonly known as nabilone.

In some embodiments, the compound is a metabolite of nabilone. Metabolites of nabilone can exist in four isomeric forms as shown below in Scheme 1.

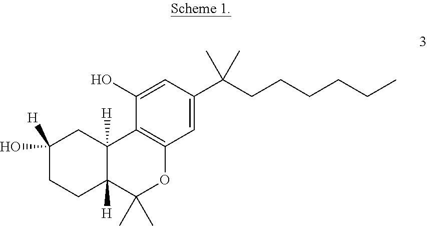 [SMILES: CCCCCCC(C)(C)c1cc(O)c2c(c1)OC(C)(C)[C@@H]1CC[C@H](O)C[C@@H]21]

### 6aR,9S,10aR)-6,6-dimethyl-3-(2-methyloctan-2-yl)-6a,7,8,9,10,10a-hexahydro-6H-benzo[c]chromene-1,9-diol

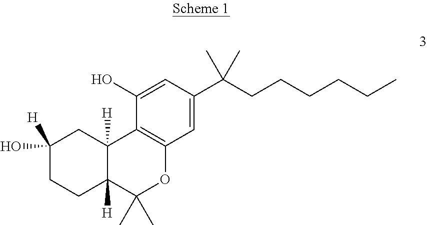 [SMILES: CCCCCCC(C)(C)c1cc(O)c2c(c1)OC(C)(C)[C@@H]1CC[C@H](O)C[C@@H]21]

### (6aR,9R,10aR)-6,6-dimethyl-3-(2-methyloctan-2-yl)-6a,7,8,9,10,10a-hexahydro-6H-benzo[c]chromene-1,9-diol

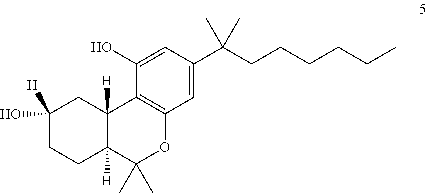 [SMILES: CCCCCCC(C)(C)c1cc(O)c2c(c1)OC(C)(C)[C@H]1CC[C@H](O)C[C@H]21]

### (6aS,9S,10aS)-6,6-dimethyl-3-(2-methyloctan-2-yl)-6a,7,8,9,10,10a-hexahydro-6H-benzo[c]chromene-1,9-diol

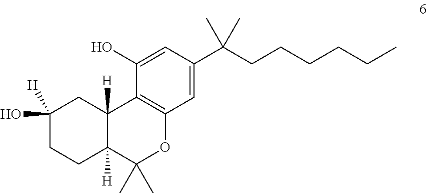 [SMILES: CCCCCCC(C)(C)c1cc(O)c2c(c1)OC(C)(C)[C@H]1CC[C@@H](O)C[C@H]21]

### (6aR,10aR)-1-hydroxy-6,6-dimethyl-3-(2-methyloctan-2-yl)-6,6a,7,8,10,10a-hexahydro-9H-benzo[c]chromen-9-one

In some embodiments, the at least one compound of Formula (I) is

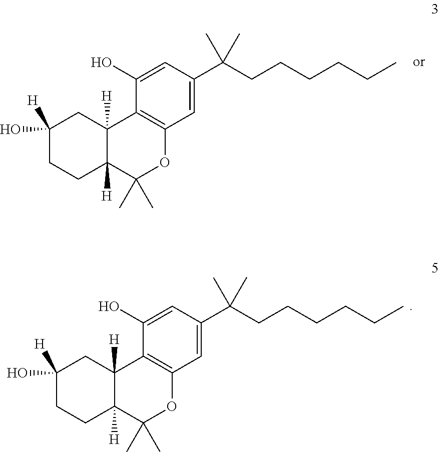 [SMILES: CCCCCCC(C)(C)c1cc(O)c2c(c1)OC(C)(C)[C@@H]1CC[C@H](O)C[C@@H]21 ; CCCCCCC(C)(C)c1cc(O)c2c(c1)OC(C)(C)[C@H]1CC[C@H](O)C[C@H]21]

Compounds 3 and 5 are metabolites of nabilone.

In some embodiments, the amount of the at least one compound of Formula (I) present in the composition is about 0.005 weight % to about 0.5 weight %, about 0.005 weight % to about 0.1 weight %, about 0.005 weight % to about 0.05 weight %, about 0.005 weight % to about 0.015 weight %, about 0.005% (w/w) to about 0.01% (w/w), about 0.01 weight % to about 0.5 weight %, about 0.01 weight % to about 0.1 weight %, about 0.01 weight % to about 0.05 weight %, about 0.01 weight % to about 0.015 weight %, about 0.015 weight % to about 0.5 weight %, about 0.015 weight % to about 0.1 weight %, about 0.015 weight % to about 0.05 weight %, about 0.05 weight % to about 0.5 weight %, about 0.05 weight % to about 0.1 weight %, or about 0.1 weight % to about 0.5 weight %.

In some embodiments, the at least one compound of Formula (I) is present in the composition at about 0.005 weight %, about 0.01 weight %, about 0.05 weight %, about 0.1 weight %, about 0.2 weight %, about 0.3 weight %, about 0.4 weight %, or about 0.5 weight %.

In some embodiments, the at least one compound of Formula (I) is present in the composition at about 0.005 weight %.

In some embodiments, the at least one compound of Formula (I) is present in the composition at about 0.015 weight %.

In some embodiments, the at least one compound of Formula (I) is present in the composition at about 0.05 weight %.

In some embodiments, the at least one compound of Formula (I) is present in the composition at about 0.075 weight %.

In some embodiments, the at least one compound of Formula (I) is present in the composition at about 0.5 weight %.

In some embodiments, the at least one compound of Formula (I) is in the dispersed phase of the composition and water is in the continuous phase of the composition.

In some embodiments, the at least one compound of Formula (I) in the dispersed phase exists as a plurality of nanosized particles or is dissolved in droplets of the oil.

In some embodiments, the at least one compound of Formula (I) in the dispersed phase exists as a plurality of nanosized particles and is dissolved in droplets of the oil.

In some embodiments, at least about 50%, about 60%, about 70%, about 80%, or about 90% of the at least one compound of Formula (I) in the composition has a mass median diameter of less than about 500 nm.

In some embodiments, at least about 90% of the at least one compound of Formula (I) in the composition has a mass median diameter of less than about 500 nm. In some embodiments, at least about 80% of the at least one compound of Formula (I) in the composition has a mass median diameter of less than about 500 nm. In some embodiments, at least about 70% of the at least one compound of Formula (I) in the composition has a mass median diameter of less than about 500 nm. In some embodiments, at least about 60% of the at least one compound of Formula (I) in the composition has a mass median diameter of less than about 500 nm. In some embodiments, at least about 50% of the at least one compound of Formula (I) in the composition has a mass median diameter of less than about 500 nm.

In some embodiments, at least about 90% of the at least one compound of Formula (I) in the composition has a mass median diameter of less than about 350 nm. In some embodiments, at least about 80% of the at least one compound of Formula (I) in the composition has a mass median diameter of less than about 350 nm. In some embodiments, at least about 70% of the at least one compound of Formula (I) in the composition has a mass median diameter of less than about 350 nm. In some embodiments, at least about 60% of the at least one compound of Formula (I) in the composition has a mass median diameter of less than about 350 nm. In some embodiments, at least about 50% of the at least one compound of Formula (I) in the composition has a mass median diameter of less than about 350 nm.

In some embodiments, at least about 90% of the at least one compound of Formula (I) in the composition has a mass median diameter of less than about 200 nm. In some embodiments, at least about 80% of the at least one compound of Formula (I) in the composition has a mass median diameter of less than about 200 nm. In some embodiments, at least about 70% of the at least one compound of Formula (I) in the composition has a mass median diameter of less than about 200 nm. In some embodiments, at least about 60% of the at least one compound of Formula (I) in the composition has a mass median diameter of less than about 200 nm. In some embodiments, at least about 50% of the at least one compound of Formula (I) in the composition has a mass median diameter of less than about 200 nm.

In some embodiments, at least about 90% of the at least one compound of Formula (I) in the composition has a mass median diameter of about 100 nm to about 200 nm. In some embodiments, at least about 80% of the at least one compound of Formula (I) in the composition has a mass median diameter of about 100 nm to about 200 nm. In some embodiments, at least about 70% of the at least one compound of Formula (I) in the composition has a mass median diameter of about 100 nm to about 200 nm. In some embodiments, at least about 60% of the at least one compound of Formula (I) in the composition has a mass median diameter of about 100 nm to about 200 nm. In some embodiments, at least about 50% of the at least one compound of Formula (I) in the composition has a mass median diameter of about 100 nm to about 200 nm.

In some embodiments, the range of the mass median diameter of the at least one compound of Formula (I) in the composition is about 1 nm to about 500 nm, about 1 nm to about 300 nm, about 1 nm to about 200 nm, about 1 nm to about 100 nm, about 1 nm to about 50 nm, about 50 nm to about 500 nm, about 50 nm to about 300 nm, about 50 nm to about 200 nm, about 50 nm to about 100 nm, about 100 nm to about 500 nm, about 100 nm to about 300 nm, about 100 nm to about 200 nm, about 200 nm to about 500 nm, about 200 nm to about 300 nm, or about 300 nm to about 500 nm.

### Oil

As used herein the term “oil” refers to any nonpolar chemical substance that is in liquid form at ambient temperature and atmospheric pressure and is both hydrophobic and lipophilic.

In some embodiments, the oil in the composition is from an animal or plant. In some embodiments, the oil in the composition is synthetic in origin.

In some embodiments, the oil in the composition is a vegetable oil.

In some embodiments, the oil in the composition is sesame oil, safflower oil, linseed oil, castor oil, a medium chain diglyceride ester oil, a medium chain triglyceride ester oil, soybean oil, olive oil, cotton seed oil, peanut oil, mineral oil, or a combination thereof.

In some embodiments, the oil in the composition is a pharmaceutically acceptable oil.

In some embodiments, the oil in the composition is sesame oil, safflower oil, castor oil, or a combination thereof.

In some embodiments, the oil in the composition is sesame oil.

In some embodiments, the oil in the composition is safflower oil.

In some embodiments, the oil in the composition is mineral oil.

In some embodiments, the oil in the composition is castor oil.

In some embodiments, the oil in the composition is MIGLYOL 812 N.

In some embodiments, the oil is present in the composition at about 1.5 weight % to about 25 weight %.

In some embodiments, the amount of oil present in the composition is about 1.5 weight % to about 25 weight %, about 1.5 weight % to about 20 weight %, about 1.5 weight % to about 15 weight %, about 1.5 weight % to about 10 weight %, about 1.5 weight % to about 5 weight %, about 5 weight % to about 25 weight %, about 5 weight % to about 20 weight %, about 5 weight % to about 15 weight %, about 5 weight % to about 10 weight %, about 10 weight % to about 25 weight %, about 10 weight % to about 20 weight %, about 15 weight % to about 25 weight %, about 15 weight % to about 20 weight %, or about 20 weight % to about 25 weight %.

In some embodiments, the oil is present in the composition at about 1.5 weight %, about 5 weight %, about 10 weight %, about 15 weight %, about 20 weight %, or about 25 weight %.

In some embodiments, the oil is in the dispersed phase of the composition and water is in the continuous phase of the composition. In some embodiments, the oil in the dispersed phase exists as a plurality of nanosized particles or droplets of the oil.

In some embodiments, at least about 50%, about 60%, about 70%, about 80%, or about 90% of the droplets of the oil in the composition have a mass median diameter of less than about 500 nm.

In some embodiments, at least about 90% of the droplets of the oil in the composition have a mass median diameter of less than about 500 nm. In some embodiments, at least about 80% of the droplets of the oil in the composition have a mass median diameter of less than about 500 nm. In some embodiments, at least about 70% of the droplets of the oil in the composition have a mass median diameter of less than about 500 nm. In some embodiments, at least about 60% of the droplets of the oil in the composition have a mass median diameter of less than about 500 nm. In some embodiments, at least about 50% of the droplets of the oil in the composition have a mass median diameter of less than about 500 nm.

In some embodiments, at least about 90% of the droplets of the oil in the composition have a mass median diameter of less than about 350 nm. In some embodiments, at least about 80% of the droplets of the oil in the composition have a mass median diameter of less than about 350 nm. In some embodiments, at least about 70% of the droplets of the oil in the composition have a mass median diameter of less than about 350 nm. In some embodiments, at least about 60% of the droplets of the oil in the composition have a mass median diameter of less than about 350 nm. In some embodiments, at least about 50% of the droplets of the oil in the composition have a mass median diameter of less than about 350 nm.

In some embodiments, at least about 90% of the droplets of the oil in the composition have a mass median diameter of less than about 200 nm. In some embodiments, at least about 80% of the droplets of the oil in the composition have a mass median diameter of less than about 200 nm. In some embodiments, at least about 70% of the droplets of the oil in the composition have a mass median diameter of less than about 200 nm. In some embodiments, at least about 60% of the droplets of the oil in the composition have a mass median diameter of less than about 200 nm. In some embodiments, at least about 50% of the droplets of the oil in the composition have a mass median diameter of less than about 200 nm.

In some embodiments, at least about 90% of the droplets of the oil in the composition have a mass median diameter of about 100 nm to about 200 nm. In some embodiments, at least about 80% of the droplets of the oil in the composition have a mass median diameter of about 100 nm to about 200 nm. In some embodiments, at least about 70% of the droplets of the oil in the composition have a mass median diameter of about 100 nm to about 200 nm. In some embodiments, at least about 60% of the droplets of the oil in the composition have a mass median diameter of about 100 nm to about 200 nm. In some embodiments, at least about 50% of the droplets of the oil in the composition have a mass median diameter of about 100 nm to about 200 nm.

In some embodiments, the range of the mass median diameter of the droplets of the oil in the composition is about 1 nm to about 500 nm, about 1 nm to about 300 nm, about 1 nm to about 200 nm, about 1 nm to about 100 nm, about 1 nm to about 50 nm, about 50 nm to about 500 nm, about 50 nm to about 300 nm, about 50 nm to about 200 nm, about 50 nm to about 100 nm, about 100 nm to about 500 nm, about 100 nm to about 300 nm, about 100 nm to about 200 nm, about 200 nm to about 500 nm, about 200 nm to about 300 nm, or about 300 nm to about 500 nm.

### Surfactant

In some embodiments, the surfactant in the composition is a nonionic, anionic, cationic, amphoteric, or zwitterionic surfactant.

In some embodiments, the surfactant is polyoxyethylene (20) sorbitan monooleate (TWEEN® 80); polyoxyethylene (20) sorbitan monolaurate (TWEEN® 20); 4-(1,1,3,3-tetramethylbutyl) phenol polymer with formaldehyde and oxirane (Tyloxapol); sorbitan monooleate (Span® 80); polyoxyethylated 12-hydroxystearic acid (KOLLIPHER® HS 15); polyoxyl 35 castor oil; polyoxyl 40 hydrogenated castor oil; polyoxyl 40 stearate; polysorbate 80; or a combination thereof.

In some embodiments, the surfactant is polyoxyethylene (20) sorbitan monooleate (TWEEN® 80) or Tyloxapol.

In some embodiments, the surfactant is polyoxyethylene (20) sorbitan monooleate (TWEEN® 80).

In some embodiments, the surfactant is polyoxyl 40 stearate, polysorbate 80, or polyoxyl 35 castor oil.

In some embodiments, the amount of surfactant present in the composition is about 0.5 weight % to about 5 weight %, about 0.5 weight % to about 2.5 weight %, about 0.5 weight % to about 1.5 weight %, about 0.5 weight % to about 1 weight %, about 1 weight % to about 5 weight %, about 1 weight % to about 2.5 weight %, about 1 weight % to about 2 weight %, about 1.5 weight % to about 5 weight %, about 1.5 weight % to about 2 weight %, or about 2 weight % to about 5 weight %.

In some embodiments, the surfactant is present in the composition at about 0.5 weight %, about 1 weight %, about 1.5 weight %, about 2.5 weight %, or about 5 weight %.

### Water

In some embodiments, the oil is in the dispersed phase and water is in the continuous phase.

In some embodiments, the ratio (w/w) of the oil to water in the composition is in the range of about 1:5 to about 1:1000 or about 1:20 to about 1:100. In some embodiments, the ratio (w/w) of the oil to water in the composition is about 1:10, about 1:30, about 1:50, about 1:70, or about 1:100.

In some embodiments, the ratio (w/w) of the oil to water in the composition is in the range of about 1:1 to about 1:1000. In some embodiments, the ratio (w/w) of the oil to water in the composition is in the range of about 1:10 to about 1:1000. In some embodiments, the ratio (w/w) of the oil to water in the composition is in the range of about 1:1 to about 1:100. In some embodiments, the ratio (w/w) of the oil to water in the composition is in the range of about 1:1 to about 1:50. In some embodiments, the ratio (w/w) of the oil to water in the composition is in the range of about 1:4 to about 1:100. In some embodiments, the ratio (w/w) of the oil to water in the composition is in the range of about 1:10 to about 1:20.

### Dispersion

In some embodiments, the composition is in the form of a dispersion. Dispersions are systems where one substance is dispersed within another substance. In some embodiments, the dispersions are heterogeneous systems where the dispersed phase, typically a particle of some type or a discontinuous liquid such as droplets of oil, is physically distinguishable from the medium in which it is dispersed. In some embodiments, the dispersion is a suspension, an emulsion, or combinations thereof. In some embodiments, the dispersion is a suspension. In some embodiments, the dispersion is an emulsion.

### Suspension

In some embodiments, the composition is in the form of a suspension.

As used herein, the term “suspension” refers to a dispersion of a solid material (the dispersed phase) in a liquid (the continuous phase) without reference to the particle size of the solid material. However, the particle size of the solid material can affect the physicochemical behavior of suspensions. For this reason, there is a distinction between a colloidal dispersion and a coarse dispersion with a colloidal dispersion having a particle size range of up to about 1 micron, and a coarse dispersion having larger particles. Pharmaceutical suspensions fall across the borderline between colloidal dispersions and coarse dispersions, with solid particles generally in the range of 0.1 to 10 micrometers. Suspensions are not optically clear and they will appear cloudy unless the size of the particles falls within the colloidal range.

### Emulsion

In some embodiments, the composition is in the form of an emulsion.

As used herein, the term “emulsion” refers to a colloidal dispersion of two or more liquid immiscible phases (or substantially immiscible phases) in the form of droplets. One of the liquid phases is normally a dispersed phase and another one is a continuous phase, wherein the dispersed phase is scattered in the continuous phase as a plurality of droplets. The emulsion can be in a form of a colloidal dispersion, a microemulsion, or a nanoemulsion based on the size of the droplets. The emulsion is an oil-in-water (o/w) emulsion if the continuous phase is an aqueous solution or a water-in-oil (w/o)-type if the continuous phase is an oil. Other examples of emulsions include oil-in-water-in-oil (o/w/o) emulsions, which comprise droplets of oil contained within aqueous droplets dispersed in a continuous oil phase.

In some embodiments, the composition is in the form of a microemulsion. A microemulsion has a droplet size in micrometers (10-6).

In some embodiments, the composition is in the form of a nanoemulsion. A nanoemulsion has a droplet size in nanometers (10-9).

In some embodiments, at least about 60% of the at least one compound of Formula (I) is present in a dispersed phase. In some embodiments, at least about 70% of the at least one compound of Formula (I) is in the dispersed phase. In some embodiments, at least about 80% of the at least one compound of Formula (I) is in the dispersed phase. In some embodiments, at least about 90% of the at least one compound of Formula (I) is in the dispersed phase. In some embodiments, at least about 95% of the at least one compound of Formula (I) is in the dispersed phase.

In some embodiments, between about 60% and about 95% of the at least one compound of Formula (I) is in the dispersed phase. In some embodiments, the percentage of the at least one compound of Formula (I) in the dispersed phase is between about 60% and about 95%, about 60% and about 90%, about 60% and about 80% about 60% and about 70%, about 70% and about 95%, about 70% and about 90%, about 70% and about 80%, about 80% and about 95%, about 80% and about 90%, or about 90% and about 95%.

### Other Components

In some embodiments, the composition further comprises at least one co-solvent. In some embodiments, the at least one co-solvent is glycerin, propylene glycol, polyethylene alcohol, ethanol, propylene glycol esters, polyethylene glycol esters, or combinations thereof.

In some embodiments, the co-solvent is glycerin.

In some embodiments, the amount of the at least one co-solvent present in the composition is about 1 weight % to about 10 weight %, about 1 weight % to about 5 weight %, about 1 weight % to about 2.5 weight %, about 1 weight % to about 2 weight %, about 1 weight % to about 1.5 weight %, about 1.5 weight % to about 10 weight %, about 1.5 weight % to about 5 weight %, about 1.5 weight % to about 2.5 weight %, about 1.5 weight % to about 2 weight %, about 2 weight % to about 10 weight %, about 2 weight % to about 5 weight %, about 2 weight % to about 2.5 weight %, or about 2.5 weight % to about 5 weight %.

In some embodiments, the co-solvent is present in the composition at about 1 weight %, about 1.5 weight %, about 2 weight %, about 2.5 weight %, about 5 weight %, or about 10 weight %.

In some embodiments, glycerin is present in the composition at about 2.5 weight %.

In some embodiments, the co-solvent is a humectant (i.e., retains moisture).

In some embodiments, the composition further comprises at least one stabilizing agent. In some embodiments, the stabilizing agent is an antioxidant.

In some embodiments, the composition further comprises at least one antioxidant. As used herein, the term “antioxidant” refers to an agent that inhibits oxidation and thus is used to prevent the deterioration of preparations by oxidation due to the presence of oxygen free radicals or free metals in the composition. In some embodiments, the at least one antioxidant is α-tocopherol acetate, butylated hydroxyanisole (BHA), Vitamin E, fumaric acid, ascorbyl palmitate, butylated hydroxytoluene (BHT), monothioglycerol, propyl gallate, sulfur dioxide, sodium thiosulfate, sodium sulfite, ascorbic acid, erythorbic acid, a bisulfite, potassium metabisulfite, malic acid, sodium metabisulfite, sodium formaldehyde sulfoxylate, or a combination thereof.

In some embodiments, the at least one antioxidant used in the composition is BHA, BHT, or a combination thereof.

In some embodiments, the concentration of antioxidant in the composition is in the range of about 0.001 weight % to about 0.5 weight %, about 0.001% weight % to about 0.1 weight %, about 0.001 weight % to about 0.05 weight %, about 0.001 weight % to about 0.01 weight %, about 0.001 weight % to about 0.005 weight %, about 0.005 eight % to about 0.5 weight %, about 0.005% weight % to about 0.1 weight %, about 0.005 weight % to about 0.05 weight %, about 0.005 weight % to about 0.01 weight %, about 0.01 weight % to about 0.5 weight %, about 0.01% weight % to about 0.1 weight %, about 0.01 weight % to about 0.05 weight %, about 0.05 weight % to about 0.5 weight %, about 0.05% weight % to about 0.1 weight %, or about 0.1 weight % to about 0.5 weight %.

In some embodiments, the concentration of antioxidant in the compositions is about 0.01 weight %, about 0.02 weight %, about 0.03 weight %, about 0.04 weight %, about 0.05%, about 0.06%, about 0.07%, about 0.08%, about 0.09%, about 0.1%, about 0.2 weight %, about 0.3 weight %, about 0.4 weight % or about 0.5 weight %. In some embodiments, the composition comprises about 0.03 weight % antioxidant (e.g., BHT and/or BHA). In some embodiments, the composition comprises about 0.03 weight % BHT and about 0.03 weight % BHA.

In some embodiments, the composition further comprises at least one pH adjusting agent. In some embodiments, the at least one pH adjusting agent is lactic acid, citric acid, phosphoric acid, acetic acid, hydrochloric acid, sodium hydroxide, potassium hydroxide, sodium carbonate, sodium hydrogen carbonate, or combinations thereof.

In some embodiments, the pH adjusting agent is sodium hydroxide or hydrochloride acid. In some embodiments, the pH adjusting agent is sodium hydroxide. In some embodiments, the pH adjusting agent is hydrochloric acid. In some embodiments, the pH adjusting agent is sodium hydroxide and hydrochloric acid.

In some embodiments, the pH adjusting agent is present in sufficient quantity to afford a pH level of about neutral. In some embodiments, the pH adjusting agent is present in a sufficient quantity to afford a pH of about 6.5 to about 7.5. In some embodiments, the pH adjusting agent is present in a sufficient quantity to afford a pH of about 6.8 to about 7.2.

In some embodiments, the composition further comprises at least one buffering agent. In some embodiments, the buffering agent is sodium diphosphate dibasic heptahydrate (NaH₂PO₄·7H₂O), sodium phosphate monobasic dihydrate (NaH₂PO₄·2H₂O), or combinations thereof. In some embodiments, the buffering agent is sodium diphosphate dibasic heptahydrate. In some embodiments, the buffering agent is sodium phosphate monobasic dihydrate. In some embodiments, the buffering agent is a combination of sodium diphosphate dibasic heptahydrate and sodium phosphate monobasic dihydrate.

In some embodiments, the composition further comprises at least one other active pharmaceutical ingredient. In some embodiments, the at least one other active pharmaceutical ingredient is suitable for ophthalmologic use. In some embodiments, the at least one other active pharmaceutical ingredient is timolol or latanoprost.

It is believed that the specific combination of components and method steps described herein impart unexpected physical, chemical, and/or microbiological stability to the compositions of the invention.

“Physically stable” compositions are those in which, for example, there is no visible phase separation between the oil phase component and the aqueous phase component under appropriate storage conditions, e.g., for at least 1, 2, 3, 4, 5, 6, 9, 12, 15, 18, or 24 months.

In some embodiments, the composition remains stable after being stored at least two years at a temperature of about 5° C., such that there is an absence of visible phase separation between the oil phase component and the aqueous phase component after such storage. In some embodiments, the composition remains stable after being stored at least one month at a temperature of about 25° C., such that there is an absence of visible phase separation between the oil phase component and the aqueous phase component after such storage.

“Chemically stable” compositions are compositions in which the concentration of the active pharmaceutical ingredient (e.g., the at least one compound of Formula (I)) does not change by more than about 20% under appropriate storage conditions for at least about two weeks or about one month.

In some embodiments, the concentration of the at least one compound of Formula (I) does not change by more than about 5%, about 10%, about 15%, or about 20% under appropriate storage conditions for at least 1, 2, 3, 4, 5, 6, 9, 12, 15, 18, or 24 months.

In some embodiments, at least about 90 weight % of the initial compound of Formula (I) in the composition remains in undegraded form after being stored for at least two years at a temperature of about 5° C. In some embodiments, at least about 90 weight % of the initial compound of Formula (I) in the composition remains in an undegraded form after being stored for at least two months at a temperature of about 25° C. In some embodiments, at least about 90 weight % of the compound of Formula (I) in the composition remains in an undegraded form after being stored for at least one month under controlled temperature conditions from about 20° C. to about 25° C.

In some embodiments, at least about 95 weight % of the initial compound of Formula (I) in the composition remains in undegraded form after being stored for at least two years at a temperature of about 5° C. In some embodiments, at least about 95 weight % of the initial compound of Formula (I) in the composition remains in an undegraded form after being stored for at least two months at a temperature of about 25° C.

In some embodiments, the compositions do not require the use of conventional preservative agents and/or excipients having antimicrobial properties to maintain microbiological stability of the compositions. In some embodiments, the compositions are substantially free of preservative agents. In some embodiments, the compositions are substantially free of benzalkonium chloride; thimerosal; chlorobutanol; methyl paraben; propyl paraben; phenylethyl alcohol; EDTA; and sorbic acid.

In addition to the advantageous physical, chemical, and microbiological stability provided by the compositions, it has also been surprisingly discovered that the compositions are highly suitable for topical administration to the eye of an animal (e.g., a human). The compositions are well-tolerated in animal studies and no irritation effects upon topical application have been detected.

The invention further provides, in some embodiments, a composition comprising: at least one compound of Formula (I);
an oil;
a surfactant;
and water,
   wherein a dispersed phase is a plurality of nanosized particles of at least one compound of Formula (I), droplets of oil, or a combination thereof, at least about 60% of the at least one compound of Formula (I) is in the dispersed phase, and water forms a continuous phase; and wherein the osmolarity of the composition is substantially similar to human tear fluid osmolarity.

As used herein, the term “osmolarity” refers to the concentration of osmotically active solutes in solution. In some embodiments, the compositions exhibit an osmolarity which is substantially similar to human tear fluid osmolarity. In some embodiments, the osmolarity of the compositions is about 300 mOsm/L to about 340 mOsm/L.

In some embodiments, the compositions are characterized in terms of their osmolality. The term “osmolality” refers to the concentration of osmotically active solutes per kg of solvent. Physiologically acceptable osmolality is osmolality in accord with the normal functioning of a living organism. Thus, for the purposes of the present invention, the osmolality of the composition is substantially similar to human tear fluid osmolality.

In some embodiments, the compositions have an osmolality of about 250 mOsm/kg to about 330 mOsm/kg. In some embodiments, the compositions have an osmolality of about 250 mOsm/kg to about 330 mOsm/kg, about 250 mOsm/kg to about 300 mOsm/kg, about 250 mOsm/kg to about 275 mOsm/kg, about 275 mOsm/kg to about 330 mOsm/kg, about 270 mOsm/kg to about 300 mOsm/kg, or about 300 mOsm/kg to about 330 mOsm/kg.

In some embodiments, the osmolality of the compositions is about 290 mOsm/kg to about 315 mOsm/kg.

The present disclosure further provides, in some embodiments, compositions comprising:
at least one compound of Formula (I);
sesame oil;
a surfactant selected from the group consisting of polyoxyethylene (20) sorbitan monooleate (TWEEN® 80); polyoxyethylene (20) sorbitan monolaurate (TWEEN® 20); 4-(1,1,3,3-tetramethylbutyl) phenol polymer with formaldehyde and oxirane (Tyloxapol); sorbitan monooleate (Span® 80); polyoxyethylated 12-hydroxystearic acid (KOLLIPHER® HS 15); polyoxyl 35 castor oil; polyoxyl 40 hydrogenated castor oil; polyoxyl 40 stearate; polysorbate 80; and a combination thereof;
and water,
   wherein the composition comprises a continuous phase comprising water; and a dispersed phase comprising a plurality of nanosized particles of at least one compound of Formula (I), droplets of the oil, or a combination thereof; and wherein at least about 60% of the at least one compound of Formula (I) is in the dispersed phase.

The present disclosure also provides a composition comprising:
about 0.005 weight % to about 0.5 weight % of the compound of Formula (I);
about 5 weight % to about 25 weight % of an oil selected from the group consisting of sesame oil, mineral oil, and castor oil;
about 1.5 weight % to about 2.5 weight % polyoxyethylene (20) sorbitan monooleate (TWEEN® 80);
about 0.01 weight % to about 1.25 weight % of BHT;
about 0.01 weight % to about 1.25 weight % of BHA;
about 2.5 weight % glycerin; and
water.

The present disclosure also provides a method of making the compositions of the invention. In some embodiments, the composition are prepared by:
combining a compound of Formula (I) and an oil to form an oil phase;
combining a surfactant and water to form an aqueous phase;
combining the oil phase and the aqueous phase to form a premix;
homogenizing the premix to form a homogenized premix; and
microfluidizing the homogenized premix to provide the composition.

In some embodiments, the homogenization of the premix occurs at a speed of about 5000 rpm for about 2 min.

In some embodiments, the homogenization of the premix occurs at a speed of about 5000 rpm for about 20 mm.

In some embodiments, the ratio (w/w) of oil to water in the premix is in the range of about 1:10 to about 1:1000 or about 1:20 to about 1:100. In some embodiments, the amount of oil in the premix is about 1.5 weight % to about 5.0 weight %.

After the homogenization step, an additional (e.g., second) portion of water can be added to form a diluted or bulk sample. The bulk sample can be filtered over a membrane to yield a composition having oil droplets of a desired size. Suitable membranes include polymer membranes having, for example, a maximum pore size of about 200 nm to about 500 nm (or about 200 nm, 250 nm, 300 nm, 350 nm, 400 nm or about 450 nm). In some embodiments, the membrane comprises a polymer material selected from polyvinylidene fluoride (PVDF), polytetrafluoroethylene (PTFE), and poly(ether sulfone) (PES).

### Particle Size

Laser diffraction is employed to measure and determine the particle size distribution of the disclosed compositions, wherein particle refers to solid particles, liquid particles such as particles dissolved in droplets of oil, or combinations thereof. Other methods exist for determining particle size distributions, but for purposes of determining whether a sample meets the particle size claim limitations, samples are analyzed by laser diffraction. A Malvern Masterizer 3000 method was employed in the examples to test the collected samples using parameters obtained from the Malvern ZetaSizer (Malvern Instruments, Malvern, United Kingdom). The particle size of the samples was analyzed by laser diffraction. Laser diffraction calculates particle size distributions by measuring the angular variation in intensity of light scattered as a laser beam passes through a dispersed particulate sample. Large particles scatter light at small angles relative to the laser beam and small particles scatter light at large angles. The angular scattering intensity data is analyzed to calculate the size of the particles responsible for creating the scattering pattern, using the Mie theory of light scattering. The particle size is reported as a volume equivalent sphere diameter.

A particle size distribution can be expressed by four values. “D”, represents the percentage of the particles in the composition that are smaller than an indicated size. The analyzer used with laser diffraction to determine particle size does not measure the particles one by one, but rather uses light with different angles and then retrieves the diffraction patterns from image sensors. Then, by performing addition, subtraction, and cross-analysis calculations, the instrument determines the statistical proportion of the sizes of the particles. The instrument also calculates the mass median diameter using software. The mass median diameter (MMD) is the particle diameter for which half of the particle mass is contained in smaller particles and half is contained in larger particles. Therefore, the MMD is half the total particle mass.

A D10 or D (10) value means that 10% of the particles in the composition are smaller than the indicated size. A D50 or D (50) value means that 50% of the particles in the composition are smaller than the indicated size. D50 is the median particle size distribution where 50% of the particles are smaller than this size and 50% of the particles are larger than this size. A D90 or D (90) value means that 90% of the particles in the composition are smaller than the indicated size. And, a D99 or D (99) value means that 99% of the particles in the composition are smaller than the indicated size.

In some embodiments, at least about 90% of the particles in the composition have a mass median diameter (MMD) wherein 50% of the mass of the dispersed droplets is qualitatively less than about 500 nm in size. In some embodiments, at least about 80% of the particles in the composition have a mass median diameter of less than about 500 nm. In some embodiments, at least about 70% of the particles in the composition have a mass median diameter of less than about 500 nm. In some embodiments, at least about 60% of the particles in the composition have a mass median diameter of less than about 500 nm. In some embodiments, at least about 50% of the particles in the composition have a mass median diameter of less than about 500 nm.

In some embodiments, at least about 90% of the particles in the composition have a mass median diameter of less than about 350 nm. In some embodiments, at least about 80% of the particles in the composition have a mass median diameter of less than about 350 nm. In some embodiments, at least about 70% of the particles in the composition have a mass median diameter of less than about 350 nm. In some embodiments, at least about 60% of the particles in the composition have a mass median diameter of less than about 350 nm. In some embodiments, at least about 50% of the particles in the composition have a mass median diameter of less than about 350 nm.

In some embodiments, at least about 90% of the particles in the composition have a mass median diameter of less than about 200 nm. In some embodiments, at least about 80% of the particles in the composition have a mass median diameter of less than about 500 nm. In some embodiments, at least about 70% of the particles in the composition have a mass median diameter of less than about 200 nm. In some embodiments, at least about 60% of the particles in the composition have a mass median diameter of less than about 200 nm. In some embodiments, at least about 50% of the particles in the composition have a mass median diameter of less than about 200 nm.

In some embodiments, at least about 90% of the particles in the composition have a mass median diameter of less than about 100 nm. In some embodiments, at least about 80% of the particles in the composition have a mass median diameter of less than about 100 nm. In some embodiments, at least about 70% of the particles in the composition have a mass median diameter of less than about 100 nm. In some embodiments, at least about 60% of the particles in the composition have a mass median diameter of less than about 100 nm. In some embodiments, at least about 50% of the particles in the composition have a mass median diameter of less than about 100 nm.

In some embodiments, at least about 90% of the particles in the composition have a mass median diameter of between about 100 nm and about 500 nm, about 100 nm and about 350 nm, about 100 nm and about 200 nm, about 200 nm and about 500 nm, about 200 nm and about 350 nm, or about 350 nm and about 500 nm. In some embodiments, at least about 90% of the particles in the composition have a mass median diameter of between about 100 nm and about 200 nm.

### Methods of Treating or Preventing

The present invention also provides a method of treating or preventing a subject in need of neuroprotection. In some embodiments, the subject is a human patient in need of neuroprotection. Neuroprotection refers to the preservation of neural tissue (such as the retina nerve or the optic nerve), and/or regeneration of the ocular nerve, and can typically be measured by a reduction of death and/or degeneration of neurons in connection with a neuropathological condition (e.g., neurological injury or disease). The neuropathic condition can include such diseases and/or disorders as blinding eye diseases, including such as, macular degeneration, retinitis pigmentosa, and glaucoma. Neuropathic conditions, such as neuropathic pain, can also be treated.

The term “subject,” as used herein, refers to a mammal, such as a human, domestic animal, such as a feline or canine subject, farm animal (e.g., bovine, equine, caprine, ovine, and porcine subject), wild animal, or a research animal (e.g., mouse, rat, rabbit, goat, sheep, pig, dog, cat, or an avian species, such as chicken, turkey, and songbird). In some embodiments, the subject is a human subject.

The present invention also provides methods of treating an ophthalmic condition in a subject by administering to the eye of the subject a therapeutically effective amount of the composition of the invention. In some embodiments, the ophthalmic condition is glaucoma, age-related macular degeneration (AMD), ophthalmitis, or conjunctivitis. In some embodiments, the ophthalmic condition is glaucoma.

In some embodiments, the ophthalmic condition is a disease of the immune system (e.g., inflammatory diseases). In some embodiments, the ophthalmic condition is a disease of the immune system such as dry eye disease, posterior uveitis, retinitis, uveoretinitis, proliferative vitreoretinopathy, anterior uveitis, episcleritis, scleritis, ocular neuropathic pain, or ocular inflammation caused by a non-infectious condition. In some embodiments, the ocular neuropathic pain can arise from dry eye, trauma, a corneal abrasion, a corneal burn, a corneal transplant, an autoimmune disease, or an allergen.

In some embodiments, the compositions provide a decrease in intraocular pressure (IOP). In some embodiments, the compositions provide a decrease in intraocular pressure for a period of at least about 1 hour after administration of the composition to the eye of the subject. In some embodiments, the compositions provide a decrease in intraocular pressure for a period of at least about 1 hour, at least about 2 hours, at least about 3 hours, at least about 4 hours, or at least about 5 hours after administration of the composition to the eye of the subject. In some embodiments, the compositions provide a decrease in intraocular pressure for a period of at least about 8 hours, at least about 12 hours, or at least about 24 hours after administration of the composition to the eye of the subject. In some embodiments, the compositions increase aqueous outflow of the eye of the subject.

As used herein, “topical administration” refers to localized administration to a surface of a tissue. For example, to an eye, particularly to any exterior aspect of the eye normally accessible between the eyelids. In some embodiments, the composition is administered topically as liquid drops, a liquid wash, a gel, an ointment, a spray, or a combination thereof. In some embodiments, the composition is in the form of an eye drop solution. For example, the composition can be presented in a rigid and/or squeeze-type bottle equipped with fitted cap constructed to serve as a dropper.

In some embodiments, the composition is administered topically as liquid drops. In some embodiments, the composition is applied as one or more drops to an ocular surface. In some embodiments, between 1 and 10 drops, between 1 and 5, between 1 and 2, between 2 and 10, between 2 and 5, or between 5 and 10 drops of the composition is administered per day.

The topical administration can further occur by way of infusing the composition via a device such as a pump-catheter system, a continuous or selective release device, a contact lens, or a combination thereof. The compositions can also be administered in injectable form, e.g., such that the composition is injected behind the eye and/or where the administration involves intravitreal injection.

In some embodiments, the compositions are administered once a day. In some embodiments, the compositions are administered twice a day. In some embodiments, the administering occurs 2 times a day, 3 times a day, 4 times a day, 5 times a day, 6 times a day, 7 times a day, 8 times a day, 9 times a day, or 10 times a day. In some embodiments, the administering occurs continuously via a release device.

Also provided herein are kits for treating or preventing an ophthalmic condition in a subject. In some embodiments, the ophthalmic condition is glaucoma. A kit can include any of the compositions described herein. The kit can include a therapeutically effective amount of the composition of the invention and can further include instructional material for administering the composition to a patient having an ophthalmic condition (such as neuropathic pain, glaucoma, age-related macular degeneration (AMD), ophthalmitis, or conjunctivitis). The instructional material can include a publication, a diagram, or any other medium of expression that can be used to communicate the usefulness of the composition and its administration. The instructional material of the kit can be attached to a container that contains the composition of the invention or can otherwise be provided together with a container that contains the composition. Alternatively, the instructional material can be provided separately, e.g., by electronic transmission, for example by means of a computer, such as by electronic mail, or download from a web site. The kit can further comprise at least one additional agent, e.g., such as an additional medicament useful for treating or preventing an ophthalmic condition.

### EXAMPLES

Having now generally described this invention, the same will be understood by reference to the following examples which are provided herein for purposes of illustration only and are not intended to be limiting unless otherwise specified.

### Example 1: Preparation of 1000 G Batch of Nabilone Nanoemulsion 0.01% (w/w)

1000 grams of a 0.01% w/w nabilone nanoemulsion was prepared using the materials shown in TABLE 3 using the following method: Obtained a 200 ml beaker and labeled it “Oil Phase.” Obtained the tare weight of the beaker. Dispensed 20.02 g of sesame oil in the “Oil Phase” beaker and heated the oil phase to 75.0±5° C. Weighed 0.1005 g nabilone and dissolved it in sesame oil, heated at 75.0±5° C. with magnetic stirring at 500 rpm. Visually confirmed that nabilone was solubilized in the oil. Obtained a 1500 ml beaker and labeled it “aqueous phase.” Added 20.03 g TWEEN® 80 and 25.05 g glycerin to the aqueous phase beaker. Added 934.91 g purified water and mixed with magnetic stirring. Heated the aqueous phase at 75.0±5° C. with magnetic stirring at 500 rpm. Gradually added the “oil phase” to the “aqueous phase” beaker and mixed for 5 minutes at a stir rate of 1000 rpm. Homogenized the mixture at 10,000 rpm for 5 minutes to form a pre-mix using a homogenizer (IKA T-18 Digital ULTRA-TURRAX®, IKA Works, Inc., Wilmington, NC). Calculated the yield after homogenization. Heated a MICROFLUIDIZER® (Microfluidics M-110P, Microfluidics, Westwood, MA) to a target temperature of 25° C. (+2° C.). Added the pre-mix to the microfluidizer and processed at 15,000 PSI. Discarded the first three strokes as waste. After processing the pre-mix for 12 minutes, the product was collected in an intermediate container. From the intermediate container, collected a 12.5 g sample (Cycle 1) for particle size evaluation. After the reservoir of the microfluidizer was drained and two strokes were processed, the contents of the intermediate container were transferred back into the reservoir and the process was repeated. After processing for 12 minutes, the product was collected in an intermediate container. From the intermediate container, a 12.5 g sample (Cycle 2) was collected for particle size evaluation. This process was repeated for three more cycles (Cycles 3, 4, and 5) with 12.5 g samples collected after each microfluidization cycle for particle size evaluation. After the fifth cycle, the product was collected and the weight was measured. Checked the pH of the emulsion and adjusted to 7.02 using 1 N NaOH followed by 1 N HCl with mixing.

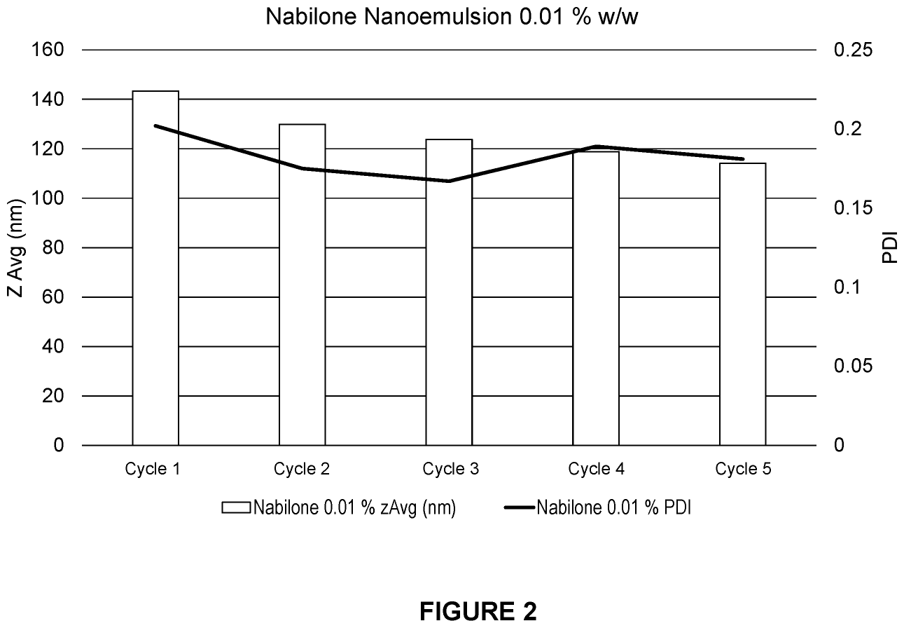

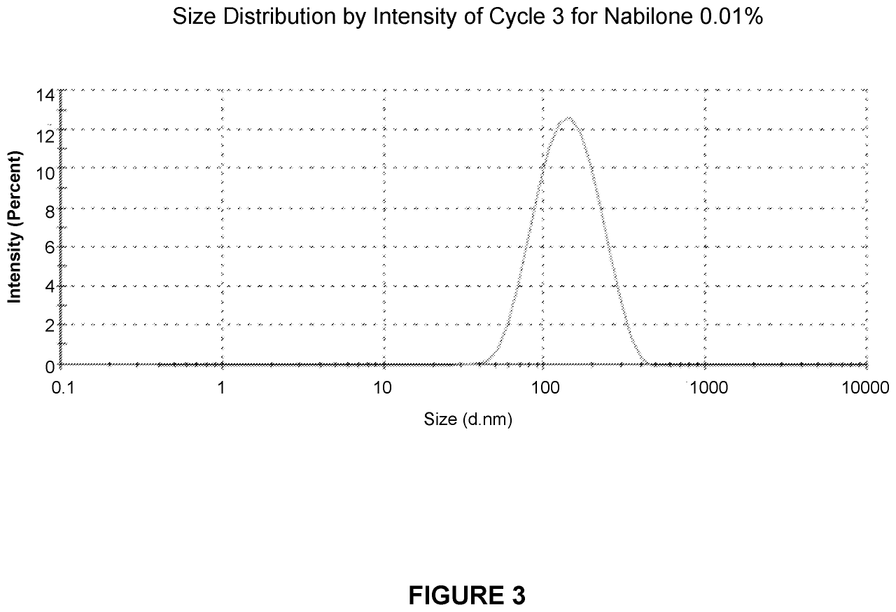

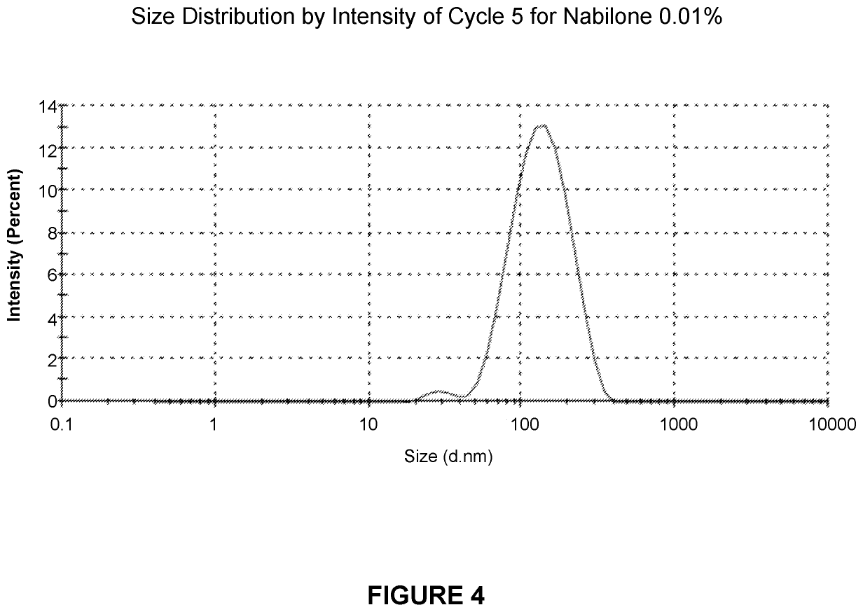

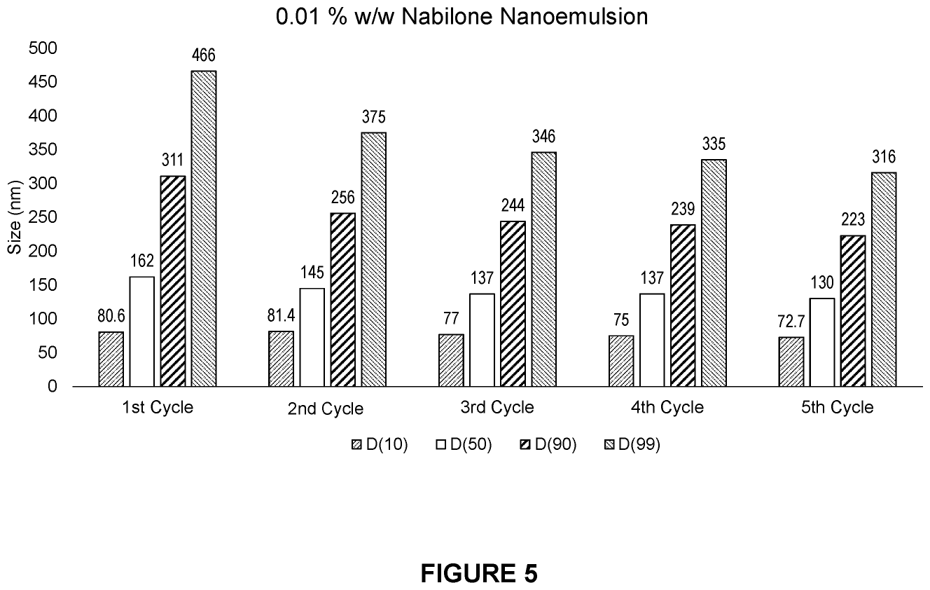

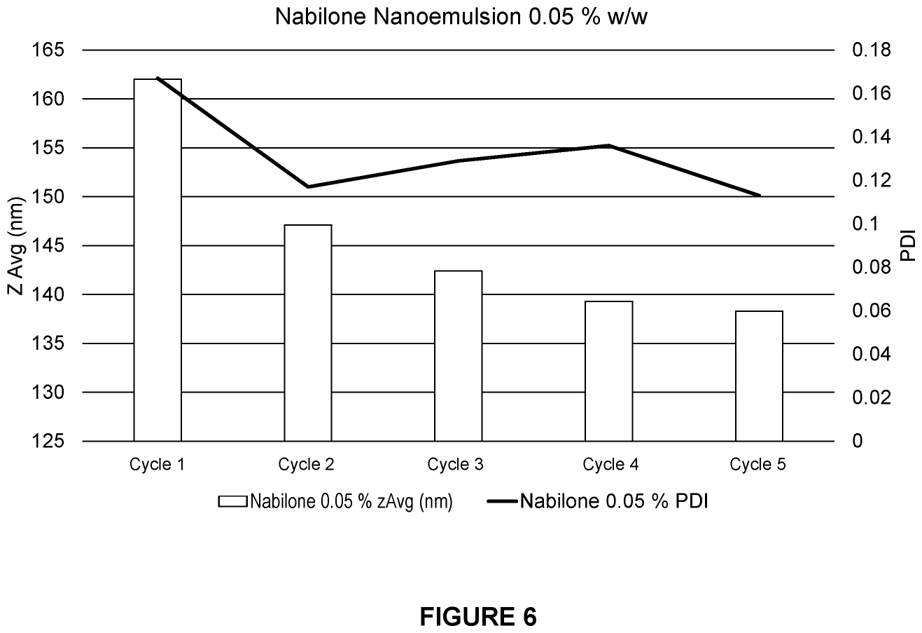

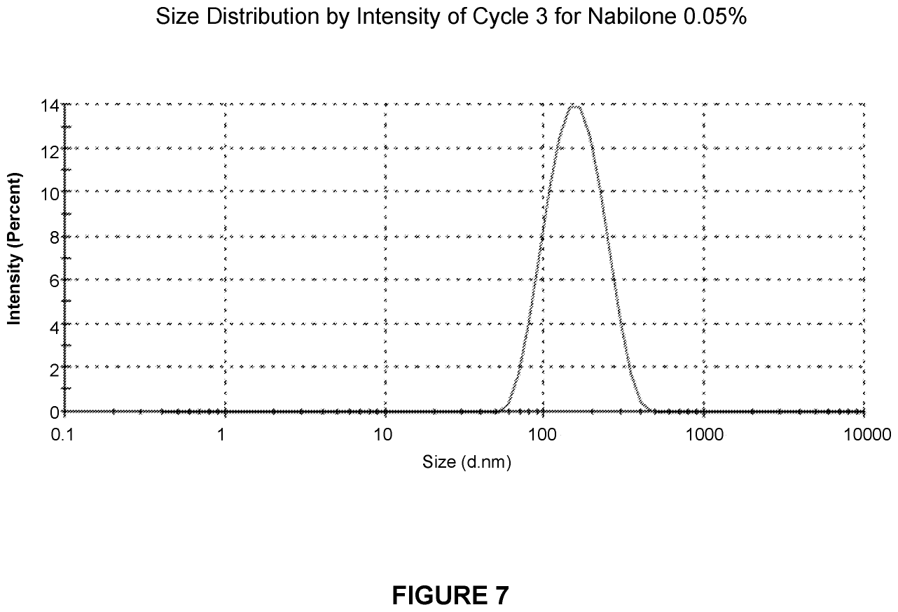

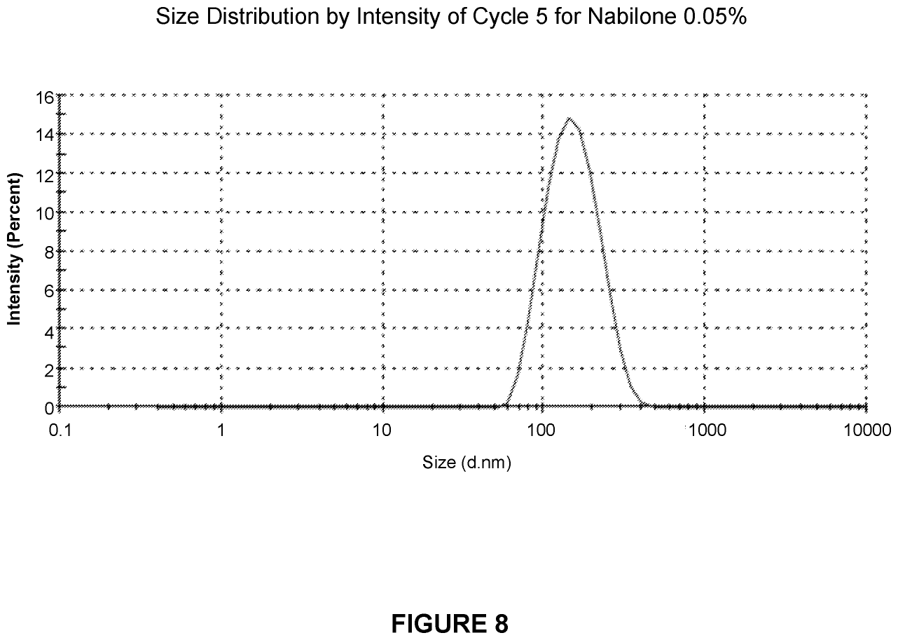

A particle size analyzer (Masterizer 3000 (Malvern Panalytical, Malvern, United Kingdom) was used for particle size distribution analysis. The particle size results obtained from the samples are shown in FIGS. 2-8.

TABLE 3
Composition of Nabilone Nanoemulsion 0.01% (w/w)

| | | Theoretical |
|---|---|---|
| | % (w/w) | wt. (g) |
| Ingredient | | |
| Nabilone | 0.01 | 0.1005 |
| Sesame Oil | 2.0 | 20.02 |
| TWEEN ® 80 | 2.0 | 20.03 |
| Glycerin | 2.5 | 25.05 |
| Purified Water | q.s. | 934.91 |
| Total | | 1000.0 |
| For pH adjustment | | |
| 1N NaOH (to adjust pH to about 7.0) | As needed | As needed |
| 1N HCl (to adjust pH to about 7.0) | As needed | As needed |

### Example 2: Preparation of 1000 G Placebo Batch to Compare to Nabilone Nanoemulsion 0.01% (w/w))

The method of Example 1 was used to prepare a placebo batch of nabilone nanoemulsion 0.01% (w/w) using the materials shown in TABLE 4 (where nabilone is absent).

TABLE 4
Placebo Batch for Nabilone Nanoemulsion 0.01% (w/w)

| | | Theoretical |
|---|---|---|
| Ingredient | % (w/w) | wt. (g) |
| Sesame Oil | 2.0 | 19.99 |
| TWEEN ® 80 | 2.0 | 20.10 |
| Glycerin | 2.5 | 25.03 |
| Purified Water | q.s. | 935.01 |
| Total | | 1000.0 |

For pH adjustment

| | | |
|---|---|---|
| 1N NaOH (to adjust pH to about 7.0) | As needed | As needed |

### Example 3: Preparation of 1000 G Batch of Nabilone Nanoemulsion 0.05% (w/w)

TABLE 5 shows to the composition of a 0.05% w/w nabilone nanoemulsion prepared using the method of Example 1.

TABLE 5
Composition of Nabilone Nanoemulsion 0.05% (w/w)

| | | Theoretical |
|---|---|---|
| Ingredient | % (w/w) | wt. (g) |
| Nabilone | 0.05 | 0.5003 |
| Sesame Oil | 10.0 | 100.03 |
| TWEEN ® 80 | 2.0 | 20.00 |
| Glycerin | 2.5 | 25.03 |
| Purified Water | q.s. | 854.52 |
| Total | | 1000.0 |

For pH adjustment

| | | |
|---|---|---|
| 1N NaOH (to adjust pH to about 7.0) | As needed | As needed |

### Example 4: Preparation of 1000 G Placebo Batch to Compare to Nabilone Nanoemulsion 0.05% (w/w)

A placebo formulation for 0.05% (w/w)) nabilone nanoemulsion was prepared using the materials using the method of Example 1. The composition of this batch is summarized in TABLE 6 (with nabilone absent).

TABLE 6
Placebo Batch for Nabilone Nanoemulsion 0.05% (w/w)

| | | Theoretical |
|---|---|---|
| Ingredient | % (w/w) | wt. (g) |
| Sesame Oil | 10.0 | 100.08 |
| TWEEN ® 80 | 2.0 | 20.01 |
| Glycerin | 2.5 | 25.07 |
| Purified Water | q.s. | 855.03 |
| Total | | 1000.0 |

For pH adjustment

| | | |
|---|---|---|
| 1N NaOH (to adjust pH to about 7.0) | As needed | As needed |

### Example 5: Preparation of 1000 G Batch of Nabilone Nanoemulsion 0.075% (w/w)

A 1000 gram batch of a 0.075% w/w nabilone nanoemulsion was prepared using the method of Example 1 and the materials shown in TABLE 7.

TABLE 7
Composition of Nabilone Nanoemulsion 0.075% (w/w)

| | | Theoretical |
|---|---|---|
| Ingredient | % (w/w) | wt. (g) |
| Nabilone | 0.075 | 0.75 |
| Sesame Oil | 15.0 | 150.0 |
| TWEEN ® 80 | 2.0 | 20.0 |
| Glycerin | 2.5 | 25.0 |
| Purified Water | q.s. | 804.25 |
| Total | | 1000.0 |

For pH adjustment

| | | |
|---|---|---|
| 1N NaOH (to adjust pH to about 7.0) | As needed | As needed |
| 1N HCl (to adjust pH to about 7.0) | As needed | As needed |

### Example 6: Preparation of 1000 G Batch of Nabilone Nanoemulsion 0.025% (w/w)

A 1000 gram batch of a 0.025% w/w nabilone nanoemulsion was prepared using the method of Example 1 and the materials shown in TABLE 8.

TABLE 8
Composition of Nabilone Nanoemulsion 0.025% (w/w)

| | | Theoretical |
|---|---|---|
| Ingredient | % (w/w) | wt. (g) |
| Nabilone | 0.025 | 0.25 |
| Sesame Oil | 5.0 | 50.0 |
| TWEEN ® 80 | 2.0 | 20.0 |
| Glycerin | 2.5 | 25.0 |
| Purified Water | q.s. | 904.25 |
| Total | | 1000.0 |

For pH adjustment

| | | |
|---|---|---|
| 1N NaOH (to adjust pH to about 7.0) | As needed | As needed |
| 1N HCl (to adjust pH to about 7.0) | As needed | As needed |

### Example 7: Preparation of Nabilone Nanoemulsion 0.075% (w/w) with Antioxidants

A 0.075% w/w nabilone nanoemulsion was prepared using the materials in TABLE 9 based on the method of Example 1 with the addition of antioxidants. Antioxidants and co-solvents can be added to pharmaceutical compositions to maintain shelf-life stability. The co-solvent (glycerin) was added to the aqueous phase as described in Example 1 and the antioxidants butylated hydroxyanisole (BHA) and butylated hydroxytoluene (BHT) were added to the oil phase before nabilone addition. The composition was shown to be stable in assay, impurities, particle size distribution, and pH at refrigerated (5° C.) and room (25° C.) temperature conditions for at least six months.

TABLE 9
Composition of Nabilone Nanoemulsion
0.075% (w/w) with Antioxidants

| | | Theoretical |
|---|---|---|
| Ingredient | % (w/w) | wt. (g) |
| Nabilone | 0.075 | 0.75 |
| Sesame Oil | 15.0 | 150.0 |
| BHA | 0.03 | 0.3 |
| BHT | 0.03 | 0.3 |
| TWEEN ® 80 | 2.0 | 20.0 |
| Glycerin | 2.5 | 25.0 |
| Purified Water | q.s. | 803.65 |
| Total | | 1000.0 |

For pH adjustment

| | | |
|---|---|---|
| 1N NaOH (to adjust pH to about 7.0) | As needed | As needed |
| 1N HCl (to adjust pH to about 7.0) | As needed | As needed |

### Example 8: Preparation of Placebo Batch of Nabilone Nanoemulsion 0.075% (w/w)

A placebo batch of nabilone nanoemulsion 0.075% was prepared using the materials in TABLE 10 based on the method of Example 1.

TABLE 10
Placebo Batch for Nabilone Nanoemulsion 0.075% (w/w)

| | | Theoretical |
|---|---|---|
| Ingredient | % (w/w) | wt. (g) |
| Sesame Oil | 15.0 | 150.0 |
| TWEEN ® 80 | 2.0 | 20.0 |
| Glycerin | 2.5 | 25.0 |
| Purified Water | q.s. | 805 |
| Total | | 1000.0 |

For pH adjustment

| | | |
|---|---|---|
| 1N NaOH (to adjust pH to about 7.0) | As needed | As needed |
| 1N HCl (to adjust pH to about 7.0) | As needed | As needed |

### Example 9: Compositions of Nabilone 0.025% (w/w) in Castor Oil Formulations

0.025% w/w nabilone nanoemulsions were prepared using the materials in TABLEs 13-15 based on the method of Example 1 substituting castor oil for sesame oil and substituting polysorbate 80, polyoxyl 35 castor oil, or polyoxyl 40 stearate for TWEEN® 80. The buffering agents sodium phosphate dibasic heptahydrate and sodium phosphate monobasic dihydrate were added to the aqueous phase.

TABLE 11
Composition of Nabilone Nanoemulsion 0.025%
(w/w) with Castor Oil and Polysorbate 80

| | % (w/w) | % (w/w) | % (w/w) |
|---|---|---|---|
| | for | for | for |
| Ingredient | pH 6.0 | pH 7.0 | pH 8.0 |
| Nabilone | 0.025 | 0.025 | 0.025 |
| Sodium Phosphate Dibasic, | 0.055 | 0.295 | 0.497 |
| Heptahydrate; Na₂HPO₄•7H₂0 | | | |
| Sodium Phosphate Monobasic, | 0.280 | 0.140 | 0.023 |
| Dihydrate; NaH₂PO₄•2H₂0 | | | |
| Glycerin | 1.531 | 1.448 | 1.379 |
| Castor Oil | 5.000 | 5.000 | 5.000 |
| Polysorbate 80 | 5.000 | 5.000 | 5.000 |
| Hydrochloric Acid/Sodium | Adjust | Adjust | Adjust |
| Hydroxide | pH 6.0 | pH 7.0 | pH 8.0 |
| Water for Injection | QS 100% | QS 100% | QS 100% |

TABLE 12
Composition of Nabilone Nanoemulsion 0.025% (w/w)
with Castor Oil and Polyoxyl 40 Stearate

| | % (w/w) | % (w/w) | % (w/w) |
|---|---|---|---|
| | for | for | for |
| Ingredient | pH 6.0 | pH 7.0 | pH 8.0 |
| Nabilone | 0.025 | 0.025 | 0.025 |
| Sodium Phosphate Dibasic, | 0.055 | 0.295 | 0.497 |
| Heptahydrate; Na₂HPO₄•7H₂0 | | | |
| Sodium Phosphate Monobasic, | 0.280 | 0.140 | 0.023 |
| Dihydrate; NaH₂PO₄•2H₂0 | | | |
| Glycerin | 1.469 | 1.386 | 1.317 |
| Castor Oil | 5.000 | 5.000 | 5.000 |
| Polyoxyl 35 Castor Oil | 5.000 | 5.000 | 5.000 |
| Hydrochloric Acid/Sodium | Adjust | Adjust | Adjust |
| Hydroxide | pH 6.0 | pH 7.0 | pH 8.0 |
| Water for Injection | QS 100% | QS 100% | QS 100% |

TABLE 13
Composition of Nabilone Nanoemulsion 0.025% (w/w)
with Castor Oil and Polyoxyl 35 Castor Oil

| | % (w/w) | % (w/w) | % (w/w) |
|---|---|---|---|
| | for | for | for |
| Ingredient | pH 6.0 | pH 7.0 | pH 8.0 |
| Nabilone | 0.025 | 0.025 | 0.025 |
| Sodium Phosphate Dibasic, | 0.055 | 0.295 | 0.497 |
| Heptahydrate; Na₂HPO₄•7H₂0 | | | |
| Sodium Phosphate Monobasic, | 0.280 | 0.140 | 0.023 |
| Dihydrate; NaH₂PO₄•2H₂0 | | | |
| Glycerin | 0.350 | 0.267 | 0.198 |
| Castor Oil | 5.000 | 5.000 | 5.000 |
| Polyoxyl 40 Stearate | 5.000 | 5.000 | 5.000 |
| Hydrochloric Acid/ | Adjust | Adjust | Adjust |
| Sodium Hydroxide | pH 6.0 | pH 7.0 | pH 8.0 |
| Water for Injection | QS 100% | QS 100% | QS 100% |

### Example 10: Compositions of Nabilone 0.025% (w/w) in Mineral Oil Formulations

0.025% w/w nabilone nanoemulsions were prepared using the materials in TABLEs 14-16 based on the method of Example 1 substituting mineral oil for sesame oil and substituting polysorbate 80, polyoxyl 35 castor oil, or polyoxyl 40 stearate for TWEEN® 80. The buffering agents sodium phosphate dibasic heptahydrate and sodium phosphate monobasic dihydrate were added to the aqueous phase.

TABLE 14
Composition of Nabilone Nanoemulsion 0.025%
(w/w) with Mineral Oil and Polysorbate 80

| | % (w/w) | % (w/w) | % (w/w) |
|---|---|---|---|
| | for | for | for |
| Ingredient | pH 6.0 | pH 7.0 | pH 8.0 |
| Nabilone | 0.025 | 0.025 | 0.025 |
| Sodium Phosphate Dibasic, | 0.055 | 0.295 | 0.497 |
| Heptahydrate; Na₂HPO₄•7H₂0 | | | |
| Sodium Phosphate Monobasic, | 0.280 | 0.140 | 0.023 |
| Dihydrate; NaH₂PO₄•2H₂0 | | | |
| Glycerin | 1.531 | 1.448 | 1.379 |
| Mineral Oil | 5.000 | 5.000 | 5.000 |
| Polyoxyl 40 Stearate | 5.000 | 5.000 | 5.000 |
| Hydrochloric Acid/ | Adjust | Adjust | Adjust |
| Sodium Hydroxide | pH 6.0 | pH 7.0 | pH 8.0 |
| Water for Injection | QS 100% | QS 100% | QS 100% |

TABLE 15
Composition of Nabilone Nanoemulsion 0.025% (w/w)
with Mineral Oil and Polyoxyl 35 Castor Oil

| | % (w/w) | % (w/w) | % (w/w) |
|---|---|---|---|
| | for | for | for |
| Ingredient | pH 6.0 | pH 7.0 | pH 8.0 |
| Nabilone | 0.025 | 0.025 | 0.025 |
| Sodium Phosphate Dibasic, | 0.055 | 0.295 | 0.497 |
| Heptahydrate; Na₂HPO₄•7H₂0 | | | |
| Sodium Phosphate Monobasic, | 0.280 | 0.140 | 0.023 |
| Dihydrate; NaH₂PO₄•2H₂0 | | | |
| Glycerin | 1.469 | 1.386 | 1.317 |
| Castor Oil | 5.000 | 5.000 | 5.000 |
| Polyoxyl 35 Castor Oil | 5.000 | 5.000 | 5.000 |
| Hydrochloric Acid/ | Adjust | Adjust | Adjust |
| Sodium Hydroxide | pH 6.0 | pH 7.0 | pH 8.0 |
| Water for Injection | QS 100% | QS 100% | QS 100% |

TABLE 16
Composition of Nabilone Nanoemulsion 0.025% (w/w)
with Mineral Oil and Polyoxyl 35 Castor Oil

| | % (w/w) | % (w/w) | % (w/w) |
|---|---|---|---|
| | for | for | for |
| Ingredient | pH 6.0 | pH 7.0 | pH 8.0 |
| Nabilone | 0.025 | 0.025 | 0.025 |
| Sodium Phosphate Dibasic, | 0.055 | 0.295 | 0.497 |
| Heptahydrate; Na₂HPO₄•7H₂0 | | | |
| Sodium Phosphate Monobasic, | 0.280 | 0.140 | 0.023 |
| Dihydrate; NaH₂PO₄•2H₂0 | | | |
| Glycerin | 0.350 | 0.267 | 0.198 |
| Castor Oil | 5.000 | 5.000 | 5.000 |
| Polyoxyl 40 Stearate | 5.000 | 5.000 | 5.000 |
| Hydrochloric Acid/ | Adjust | Adjust | Adjust |
| Sodium Hydroxide | pH 6.0 | pH 7.0 | pH 8.0 |
| Water for Injection | QS 100% | QS 100% | QS 100% |

### Example 11: Mouse Study on IOP Lowering Effects

### Animals

Male C57BL/6J mice (Jackson Laboratories, Bar Harbor, ME; age 8 months) were kept in 12 h light/12 h dark conditions (lights on 0600 h) and fed with standard chow. All experimental procedures were conducted in accordance with the ARVO Statement for the Use of Animals in Ophthalmic and Vision Research, and the University of North Texas Health Science Center Institutional Animal Care and Use Committee Regulations and Guidelines.

### Ophthalmic Formulations

Nabilone nanoemulsions (0.01, 0.025, 0.05, and 0.075 weight %) were prepared as described in Examples 1, 3, 5, and 6. Control placebo nanoemulsions (for comparison with 0.01, 0.025, 0.05, and 0.075 weight % nabilone nanoemulsions) were prepared as described in Examples 2, 4, and 8. Timolol maleate (0.5 weight %) was purchased from Sandoz Inc., Princeton, NJ (A Novartis Division) (Manufactured for Sandoz Inc. by Alcon Laboratories Inc., Fort Worth, TX).

### IOP Measurements

IOP was determined in behaviorally trained conscious animals using a TONOLAB® rebound tonometer (Colonial Medical Supply, Franconia, NH), in accordance with the procedure set forth in Wang et al., Invest Ophthalmol Vis. Sci., 2005, 46:4617-4621. The indicated formulation was topically administered to the left eye of each animal. The contralateral eye was not treated.

The animals were divided into four groups (as specified below). The baseline IOP of both eyes at −1 hour prior to drug treatment was measured, and direct ophthalmoscopy was performed. Next, a single 5 μL drop was instilled topically onto the left eye of each mouse. The contralateral (right) eye was untreated.

The TOP of both eyes was measured again at 2 hours, 4 hours, 6 hours, 24 hours, 30 hours, and 48 hours after dosing. In addition to IOP measurement, animals were evaluated by direct ophthalmoscopy for possible ocular and gross systemic adverse effects.
Group 1: Nabilone (0.01 weight %)
Group 2: Nabilone (0.05 weight %)
Group 3: Control for nabilone (0.05 weight %)
Group 4: Timolol (0.5 weight %)

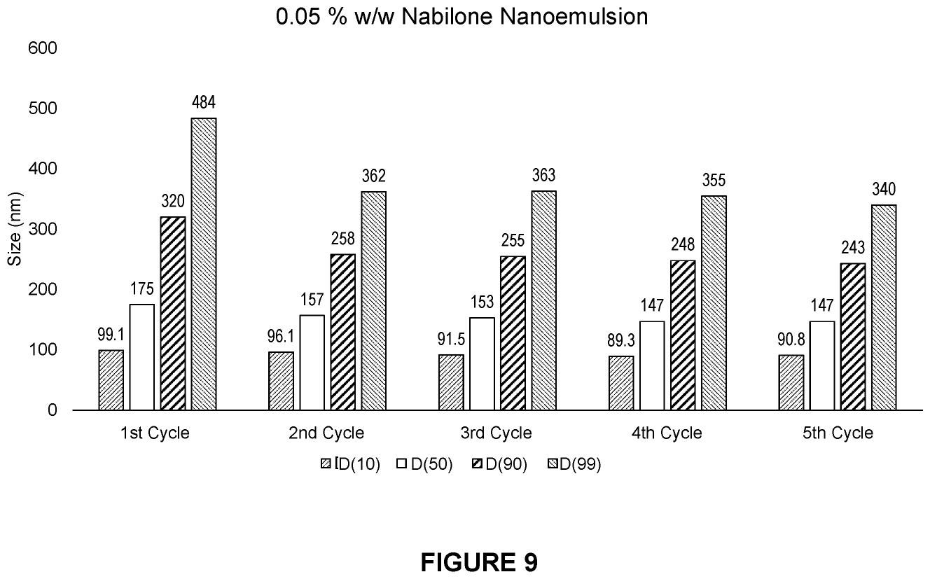

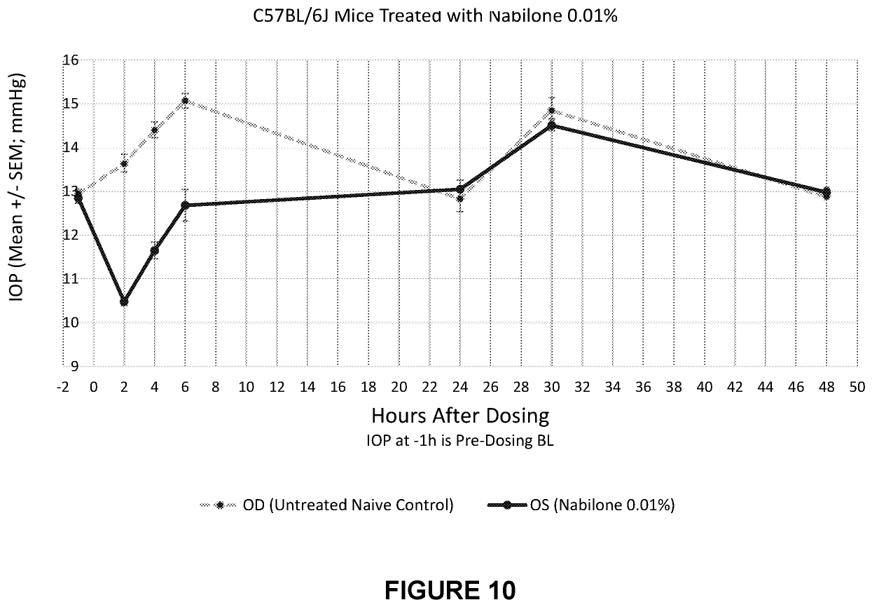

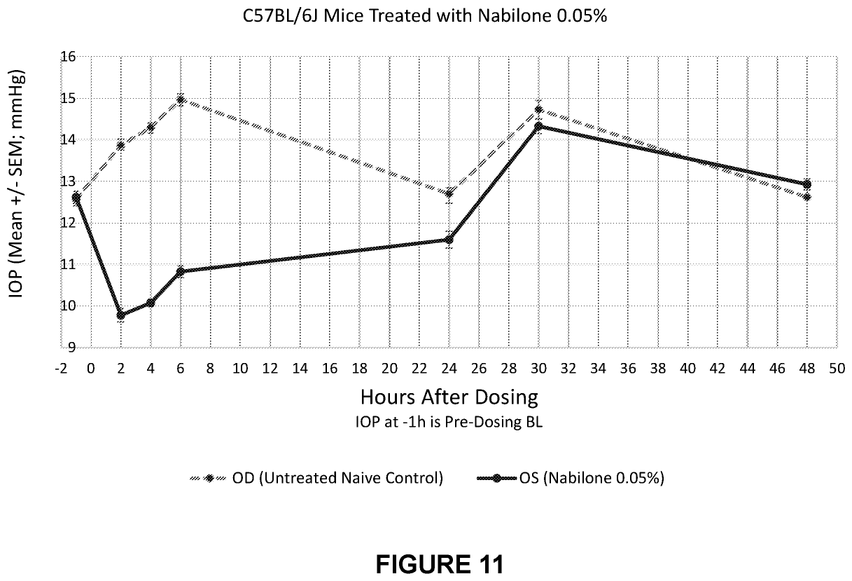

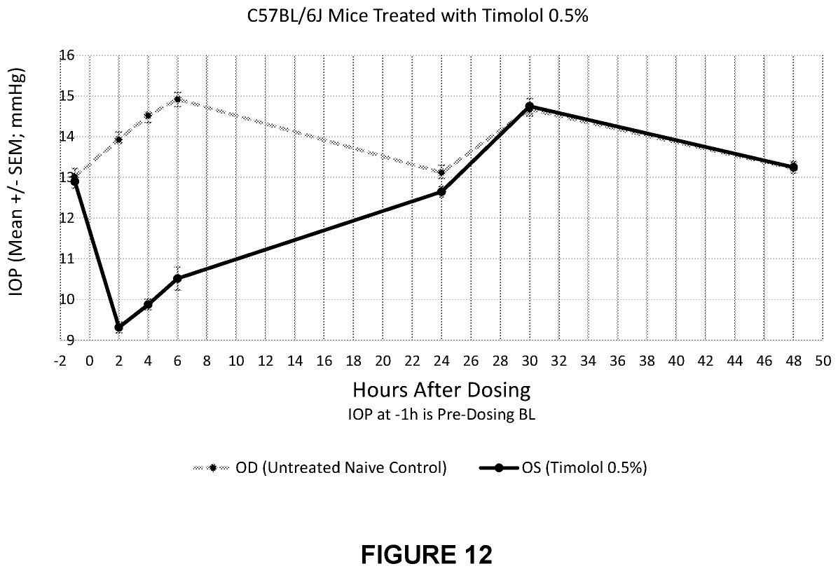

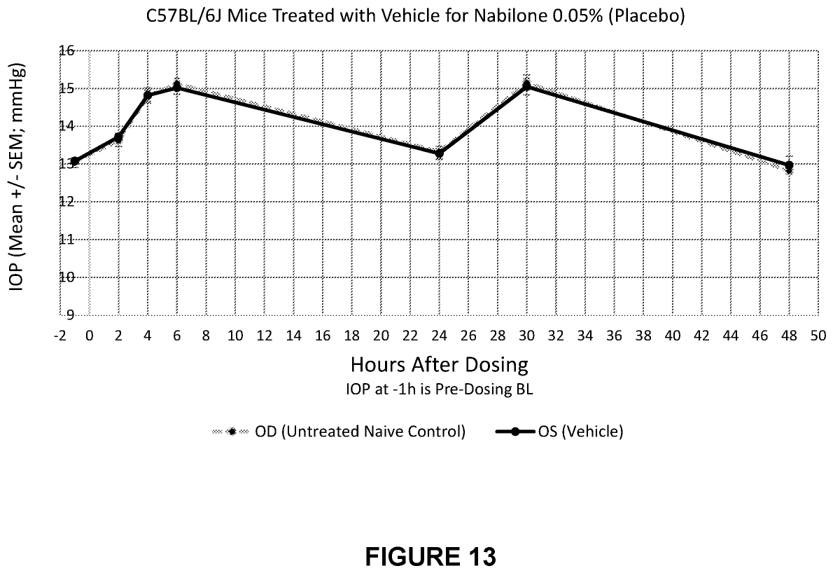

Results are presented in FIGS. 9-13, which illustrate comparison of IOP effects of the nabilone compositions (0.01 weight % and 0.05 weight %), the Control composition for nabilone (0.05 weight %), and Timolol. The compositions were administered once at 9-10 AM (Time 0), immediately following baseline IOP measurements. Data are shown as mean±SEM (standard error of the mean).

The formulation effect was also studied with nabilone nanoemulsions of 0.025 and 0.075 weight % as follows:
Group 1: Nabilone (0.025 weight %)
Group 2: Nabilone (0.075 weight %)
Group 3: Control for nabilone (0.075 weight %)
Group 4: Timolol (0.5 weight %)

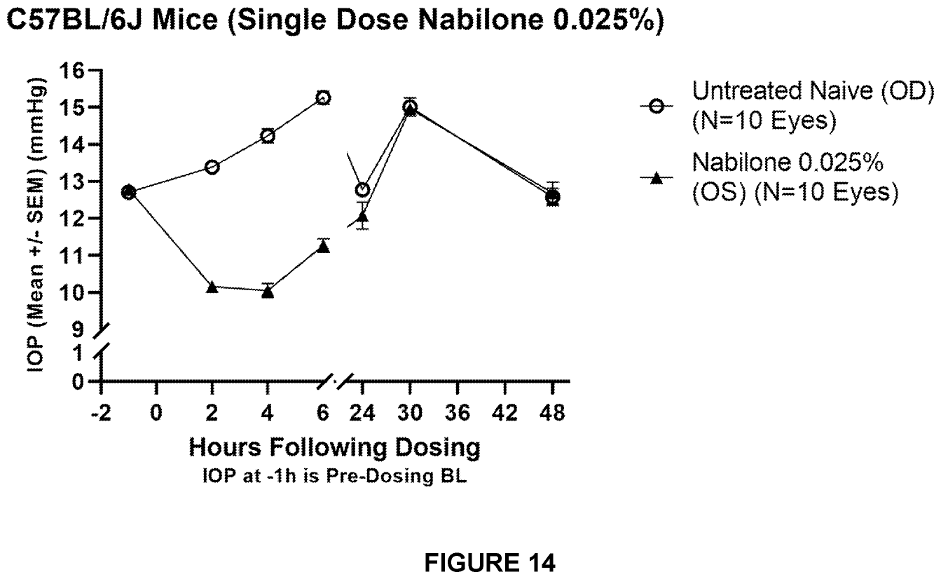

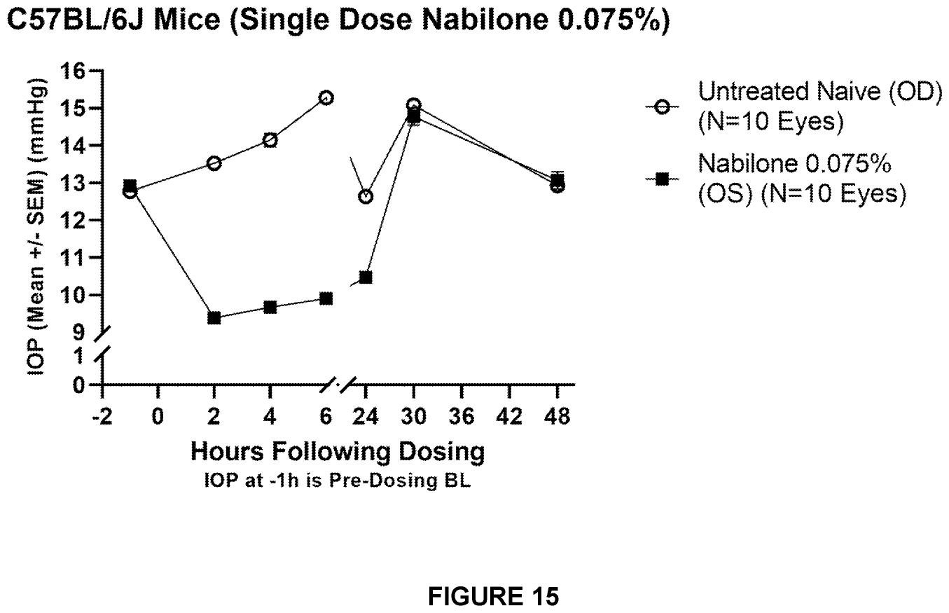

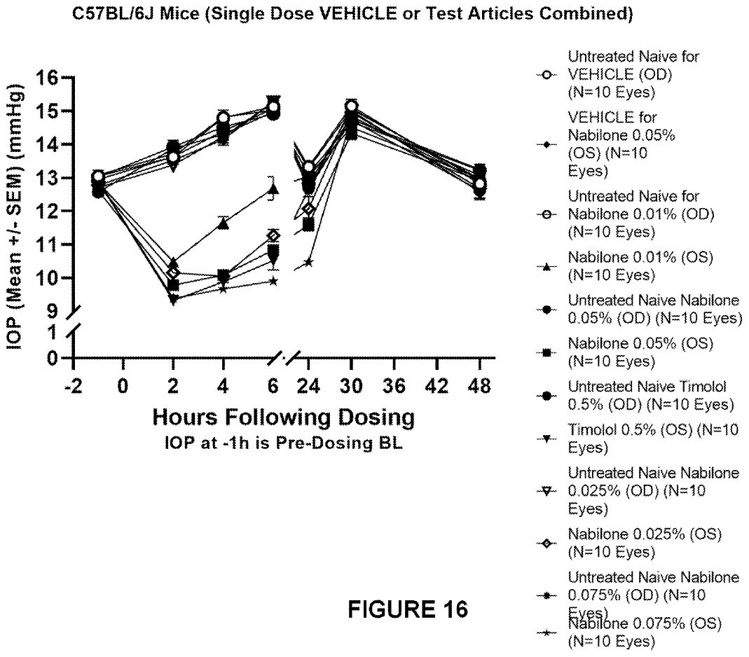

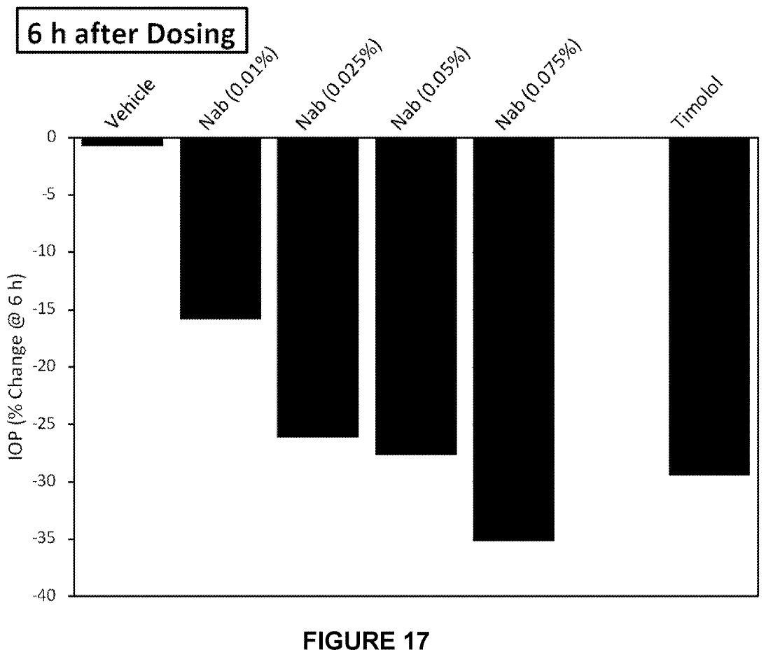

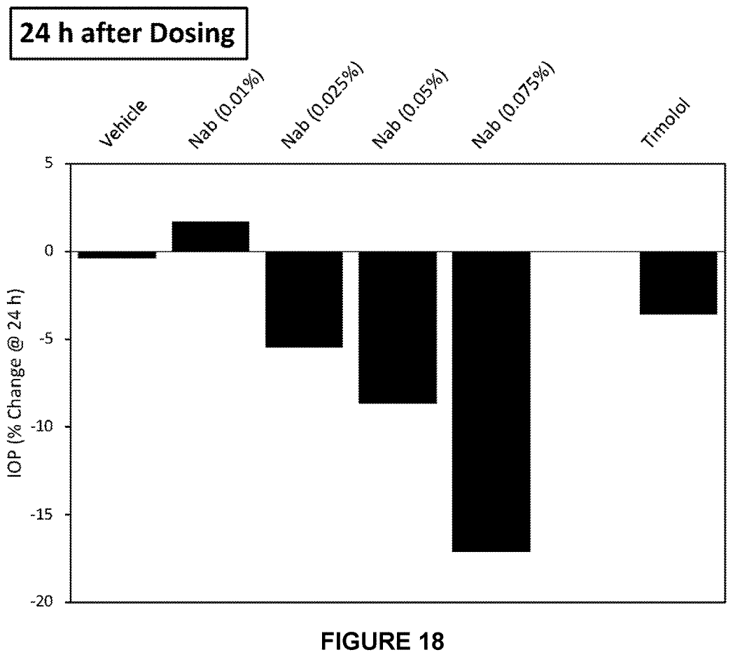

Results are presented in FIGS. 14 and 15. The combined dose response of 0.01, 0.025, 0.05, and 0.075 weight % nabilone nanoemulsions is shown in FIGS. 16, 17, and 18.

All tested nabilone compositions (with 0.01, 0.025, 0.05, and 0.075 weight % of nabilone API) produced significant intraocular pressure (IOP) reduction. No tachyphylaxis or adverse effect was observed during the 48 hours.

Compared to the contralateral untreated eyes, the eyes treated with the placebo vehicle did not lower IOP. Nabilone formulations (0.01, 0.025, 0.05, and 0.075 weight %) afforded IOP reduction in treated eyes with dose-dependent response. Nabilone formulations (0.05% and 0.075%) afforded IOP reduction comparable to that of Timolol (0.5%) in early hours from dosing. Nabilone formulations (0.05% and 0.075%) maintained IOP lowering up to 24 hours from dosing while IOP lowering by other formulations diminished.

It is to be appreciated that the Detailed Description section, in addition to the Summary and Abstract sections, is intended to be used to interpret the claims. The Summary and Abstract sections may set forth one or more but not all exemplary embodiments of the present invention as contemplated by the inventor, and thus, are not intended to limit the present invention and the appended claims in any way.

The breadth and scope of the present invention should not be limited by any of the above-described exemplary embodiments, but should be defined only in accordance with the following claims and their equivalents.

All publications, patents, and patent applications mentioned in this specification are herein incorporated by reference to the same extent as if each individual publication, patent, or patent application was specifically and individually indicated to be incorporated by reference.

1. A composition suitable for application to an ocular surface, comprising:
at least one compound of Formula (I) 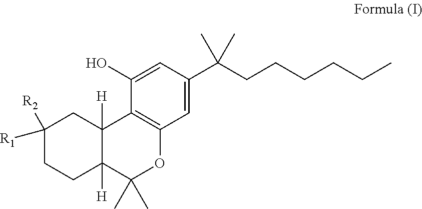
where R₁ and R₂ together form ═O or R₁ is —OH and R₂ is —H;
an oil;
a surfactant; and
water,
wherein the composition comprises a continuous phase comprising water; and a dispersed phase comprising a plurality of nanosized particles of at least one compound of Formula (I), droplets of the oil, or a combination thereof, and wherein at least about 60% of the at least one compound of Formula (I) is in the dispersed phase.

2. The composition of claim 1, wherein composition is in a form of a dispersion; a suspension; an emulsion; or any combination thereof.

3. The composition of claim 1, wherein the compound of Formula (I) is selected from the group consisting of:

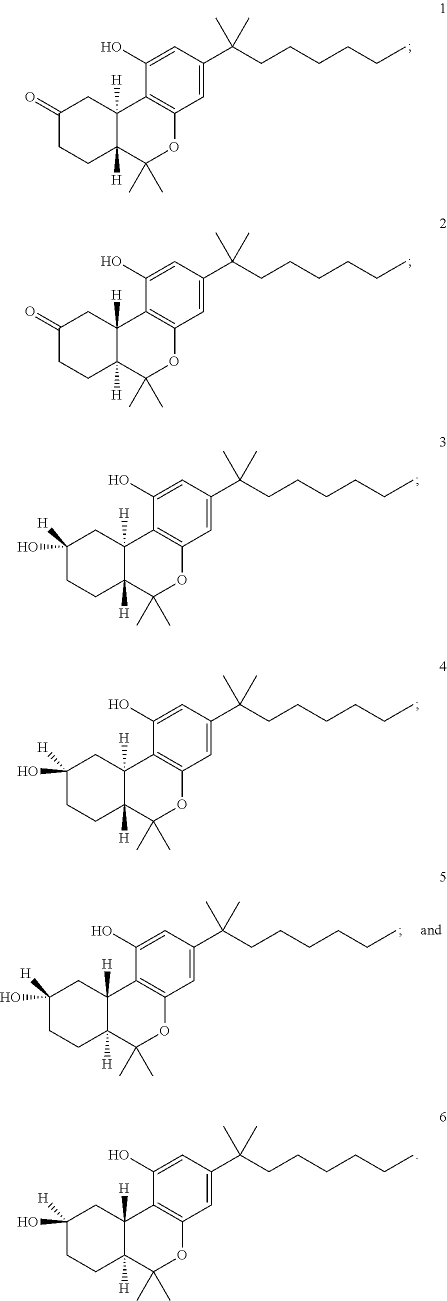 [SMILES: CCCCCCC(C)(C)c1cc(O)c2c(c1)OC(C)(C)[C@@H]1CCC(=O)C[C@@H]21 ; CCCCCCC(C)(C)c1cc(O)c2c(c1)OC(C)(C)[C@H]1CCC(=O)C[C@H]21 ; CCCCCCC(C)(C)c1cc(O)c2c(c1)OC(C)(C)[C@@H]1CC[C@H](O)C[C@@H]21 ; CCCCCCC(C)(C)c1cc(O)c2c(c1)OC(C)(C)[C@@H]1CC[C@@H](O)C[C@@H]21 ; CCCCCCC(C)(C)c1cc(O)c2c(c1)OC(C)(C)[C@H]1CC[C@H](O)C[C@H]21 ; CCCCCCC(C)(C)c1cc(O)c2c(c1)OC(C)(C)[C@H]1CC[C@@H](O)C[C@H]21]

4.-5. (canceled)

6. The composition of claim 1, wherein the compound of Formula (I) is a racemic mixture of

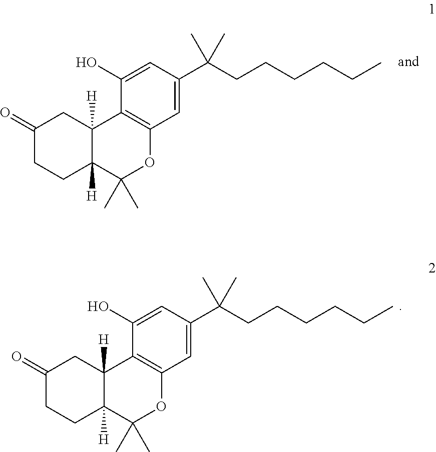 [SMILES: CCCCCCC(C)(C)c1cc(O)c2c(c1)OC(C)(C)[C@@H]1CCC(=O)C[C@@H]21 ; CCCCCCC(C)(C)c1cc(O)c2c(c1)OC(C)(C)[C@H]1CCC(=O)C[C@H]21]

7. (canceled)

8. The composition of claim 1, wherein at least about 90 weight % of the at least one compound of Formula I in the composition is present after the composition is stored at a condition selected from the group consisting of at least two years at about 5° C.; and at least one month at about 25° C.

9. The composition of claim 1, wherein the oil and water are present in the composition in a ratio (w/w) of about 1:1 to about 1:1000.

10.-11. (canceled)

12. The composition of claim 1, wherein at least about 90% of the droplets of the oil in the composition have a mass median diameter of less than about 500 nm.

13-14. (canceled)

15. The composition of claim 1, wherein at least about 90% of the at least one compound of Formula (I) in the composition has a mass median diameter of less than about 500 nm.

16.-17. (canceled)

18. The composition of claim 1, wherein the composition comprises about 0.005 weight % to about 0.5 weight % of the at least one compound of Formula (I).

19. The composition of claim 1, wherein the oil is selected from the group consisting of sesame oil, safflower oil, linseed oil, castor oil, a medium chain diglyceride ester oil, a medium chain triglyceride ester oil, soybean oil, olive oil, cotton seed oil, peanut oil, mineral oil, and combinations thereof.

20.-23. (canceled)

24. The composition of claim 1, wherein the composition comprises about 1.5 weight % to about 25.0 weight % oil.

25. The composition of claim 1, wherein the surfactant is selected from the group consisting of polyoxyethylene (20) sorbitan monooleate (TWEEN® 80); polyoxyethylene (20) sorbitan monolaurate (TWEEN® 20); 4-(1,1,3,3-tetramethylbutyl) phenol polymer with formaldehyde and oxirane (Tyloxapol); sorbitan monooleate (Span 80); polyoxyethylated 12-hydroxystearic acid (KOLLIPHER® HS 15); polyoxyl 35 castor oil; polyoxyl 40 hydrogenated castor oil; polyoxyl 40 stearate; polysorbate 80; and combinations thereof.

26. (canceled)

27. The composition of claim 1, wherein the composition comprises about 0.5 weight % to about 5 weight % surfactant.

28.-29. (canceled)

30. The composition of claim 28, wherein the composition further comprises from about 1 weight % to about 10 weight % of a co-solvent.

31.-34. (canceled)

35. The composition of claim 1, further comprising a stabilizing agent.

36.-38. (canceled)

39. The composition of claim 1, wherein the composition has an osmolarity of about 250 mOsm/L to about 330 mOsm/L.

40. (canceled)

41. The composition of claim 1, wherein the composition comprises:
about 0.005 weight % to about 0.5 weight % of the at least one compound of Formula (I);
about 5 weight % to about 25 weight % oil, wherein the oil is selected from the group consisting of sesame oil, castor oil, mineral oil, and combinations thereof;
about 1.5 weight % to about 2.5 weight % surfactant, wherein the surfactant is polyoxyethylene (20) sorbitan monooleate (TWEEN® 80);
about 0.01 weight % to about 1.25 weight % of BHT;
about 0.01 weight % to about 1.25 weight % of BHA;
about 2.5 weight % glycerin; and
water.

42. A method of treating or preventing an ophthalmic condition in a subject identified in need of such treatment, the method comprising administering to at least one eye of the subject in need thereof, a therapeutically effective amount of the composition of claim 1.

43.-44. (canceled)

45. The method of claim 42, wherein the composition is administered topically.

46.-47. (canceled)

48. The method of claim 42, wherein the ophthalmic condition is selected from the group consisting of glaucoma, age-related macular degeneration (AMD), ophthalmitis, and conjunctivitis.

49. (canceled)

50. The method of claim 42, wherein the method decreases intraocular pressure (IOP) for a period of at least about 1 hour after administering the composition to the eye.

51. (canceled)

52. A method of preparing the composition of claim 1, comprising:
combining the compound and the oil to form an oil phase;
combining the surfactant and water to form an aqueous phase;
combining the oil phase and the aqueous phase to form a premix;
homogenizing the premix to form a homogenized premix; and
microfluidizing the homogenized premix to provide the composition.

53.-60. (canceled)
